SEQUENCE LISTING (32 sequences)

SEQ ID NO: 1 - protein, 167 aa, Artificial Sequence
  MO-1
MYMGMMCTAKKCGIRFQPPAIILIYESEIKGKIRQRIMPVRNFSKFSDCTRAAEQLKNNPRHKSYLEQVSLRQLEKLFSFLRGYLSGQSLAETMEQIQRETTIDPEEDLNKLDDKELAKRKSIMDELFEKNQKKKDDPNFVYDIEVEFPQDDQLQSCGWDTESADEF

SEQ ID NO: 2 - DNA, 1400 bp, Artificial Sequence
  MO-1
cgctcgccgggacctggaatccctgtacgccgaggtgggagccggtggaccggtcccccagccggcccccacctccgcttcccggtgtttgagggttcgggcctcccgccggggagttcacccctcgggctcgtcagtagggctgtggctgtcgcctcttcctgcagcgccaggctccgcccggtctcacagtcggcttaggggctttgcgtgcactgcggttgggtggaaaaacccactcctggttgtttagacgttggcctgcagacgatgtcatttctgtattcctctaaggcaggaagtcattatgcaacttacacatattcatcagatttcctctgacttacccggacatgtacgtgggaatgatgtgcactgccaagaaatgtgggattaggtttcagcctccagctattatcttaatctatgagagtgaaatcaaggggaaaattcgccagcgcattatgccagttcgaaacttttcaaagttttcagattgcaccagagctgctgaacaattaaagaataatccgcgacacaagagttacctagaacaagtatccctgaggcagctagagaagctattcagttttttacgaggttacttgtcggggcagagtctggcagaaacaatggaacaaattcaacgggaaacaaccattgatcctgaggaagacctgaacaaactagatgacaaggagcttgccaaaagaaagagcatcatggatgaactttttgagaaaaatcagaagaagaaggatgatccaaattttgtttatgacattgaggttgaatttccacaggacgatcaactgcagtcctgtggctgggacacagagtcagctgatgagttctgataccaaacactcaaaacatgcattgggctagcagaatatccatgtttattaccagactggttctggaagaagctgtaaagaatactaaatatgttgggttataggggattgaccatgttacttttcaaaaccaggacatttaaagcatctactatgtaggtgcatgaggagtatgggaaaaacagaataaaggaatctgcctttaaggagcttacaatcatgccgggtgcggtggctcacgcctgtaatcccagcactttgggaggctgaggcgggtggatcacctaaggtcaggagttcgagaccagcctagccaacatggtgaaacctcgcctctactaaaaatacaaaaattagccaggcgtggtggcgggtgcctgtaatcccggctactcaggaggctgaggcaggagattcgcttgaacctgggaggctgaagttgcagtgagccgagatcgcgccattgtactccagcctgggcgatgagcaaaactccatctcaaaaaaaaaaaaaaaaaaaaaaaaaaaaaaaaaaaaaaaaaa

SEQ ID NO: 3 - DNA, 7176 bp, Artificial Sequence
  MO-1
actaaacggatctaaatgataccaaggatgacagaaacatcctcattttacagatggagaaacttgtagggtttgtgctttcctcccatcaaaccttaccctttattgtaattattcccgcacttgcccgtctcagtagtacaagcagtttgccaactactacttgcacgaacaactagcagtttgccacggagtcctacattcagtttggggattctcaggcctcacagggcgctaaggccttgcctggccccttggtgatcagaagcttcgtgcagctcaagagcatcccaagacgcttgacggctgactttccttctcagaactttagtaacagggcggccagtccagcctgggaaccgcagggcccgagcccgactctcccggagacccgggatccgccgcagagcaaagcgcacgggagggaaggagggcacacggctctagtgtctgacctcctccggctcgccctactgatctaggtcccgcgccgggtccccacatccccgcaacccgccgagaggcccgggagccgggaccgctcccacgctctggccccccaagccccgccccccttaccggcgtcagagcgcgcggcggtgaaggccgcggcgggcccgcgcgtgagcccggtcgccgccccgaggagcagccaggcggccgcccgaaccgccgccacccgagccgccaggacgccggaagccggacgcccgagaggggcgcgcggggccaggtgcggacgcaggaaggggcctggccgcccgcagctcctaccgcgcgctgccccagatttatcggctgggaccgagtgctgggtgcgcgcgcctagcgcccgcggttcccagctgcctgcagccccggcccccaagggttcccgcgcggcgtggggcggtcgtccccgcccagctgttccttgctccgcccacccgggggcgggcgaggagctgcgcacgcgcggggacgcgcgcccggcctgtcgctgtggaaaccgctaggccagcgctcgccgggacctggaatccctgtacgccgaggtgggagccggtggaccggtcccccagccggcccccacctccgcttcccggtgtttgagggttcgggcctcccgccggggagttcacccctcgggctcgtcagtagggctgtggctgtcgcctcttcctgcagcgccaggctccgcccggtctcacagtcggcttaggggctttgcgtgcactgcggttgggtggaaaaacccactcctggttgtttagacgttggcctgcagacgatgtcatttctgtattcctctaagggtaagaaagccagccttgcactagtggagacgtgagtagggagagaatcgtctactctttggggacattcgtacccttaggtgctgtggggcttcctaacaagattgtgggcagttactgatctctggggagaggagtattcgtgtccaggaagaataattggataaacgtgccgtccttcttcctaaacagagtggaatctgtaagcaggccaagagagcatccaggcggcttggagtggaatagagggctctagagcgggtgtctcaaagaggaaacagagtgtatgtcaaaacatactatccaaaactgaaaatagccgggcaggtggctcacgcctatagccccagcactttgggcgaatcacctgaggtcaggagttcgagaccagcctggccaagatggcgaaaccccgtctctactgaaaatacaaagattagctgggcatggtggtaggcacctgtaattccagctactcgggaggctgaggcacgagaatcgcttgaacccaggaggcggaggttgcagcgaggggagatcacgccactgcactgcagcctaggcgacagagggagactctgtttcaaaagaaaaaaaaaaactgaaaatggtagtataagcatgtttcttaagaaatggagagagaaattttggaagatatgccttttaaaattcagtctggctccctatgaagaggagtagggccaaggacgatagtttgttgttataagcctcatagaattattttatattccaaatacaaacattactttagcaaaaattaaagttaaattttaaaaagagtctagtaaaaaaaaaactacattatttgagtaccaactgcatacatctttgacgagtacagccattgcttcagtgtgtggagtttatatgtgccccgtaggttgtagtttactaacatcaaattttagttaggtgaattgggaatagtattcgttaaaaaaaaaaaaaaagcaagaaaagagaaggaaaagaaggagaaatggtgtgagctcattgcatcagattagaggtagagcacagtaattttttaaagtagttttttttttttttttttttttttttaagaataggcgtatcatgctttggagaggctgttgtgcagtggcaccatctcaactcactgcagccccaaactcctgggttcaggcgattctcccaccttggcctttggtgtggctgggactacaggtgcatgctgccatgcctggccaatttttaaattttttgtagagacatgcatggtctcgctgtgttgcccaggctggtctcaaactcctggcctcaaggtatctcctctccttggcctcccaaagtgttgggattacaggcctgagccaccacacctgcctccatctttttgagatggagtcttgctcttgtcacccatgctggagtgcagtggtaccatctcagctcactgcaacctccgcctcccaggttcaagtgattctcctgccttggcctctcgagtagctgggattacaggcacctgccactatgtccagctaatttttgtatttttagtacagacgtggttttcaccatgttggccaggctggtctcgaactcctgacctcaaatgatctgcccaccttgacctcccaaagtgctggctggggtgagccactgtgcctgggcttatatgtgattaattttctaagagtaaaaattctcatgcccaacaataagtggtgaataatatgattatagtacatccataaaataaaacatttagcagtcatataaaatgttacattgtagtaaaacatttgatgacaaaataataatatatgttgttaaatttgaacgtcaggaaatagcttgtatgataaggtatgtatgaaacatactataaactatatgcctagaaaaaagactggacacaatcaagggtgattattttctataggtttcccctctcctttgcccccagtattctacaatgacgtgtataatcactgctaagtatttaataaaaagtatgttcctaatagtgattctccagcagaactgagttcacgaagatatcatcaccctatgccactttacctcaatgtttcttgacatttacaaatctttctgggaatgtcacagtcacatcaaaagcagtgcttagggttaagtgtattctttgggcactgagggtggggaaaggaagagagaatagtaagtattcatctacaaatagctgtttcttgtagctagaaaattatcatatagtgagacacacaggccaaaataaaagccctgcagacattactgactattgtatatgaagaagctgagatatgtgatgtatatgatgtagctgcagacgttactgacgtgtatatgaagaagctgattctgcagcaggtctgctttttgatcaggcttctagactgtaaatgctttcaaaaatgtacagggaccatcatgttaattcatagcattaaacaatgtatggaatgcccactacgttccaggtattatgctgagatattgtggtggaaaatgaatatgccccccaccctcatggagcatattgactagaagggaagatagatgattaaataaataatgacaaaaaactctagtaaaataattgggaaagtacaaaggagacagttctggattggtgaacctctcaggacaaaatgattaaggttaaaaaacttaccttatcatagtaccttgataccgaggatgctcgatatgccctttcttaattgataaatagtaaagaccctataggtcagctattgggctgacttaggggttcatattcgatcttcagtaagagtgcagtacactaagaggttcaaactggattgcatccccagattcacatatgaacaagtctatatccttgaaaaaaccagatcttagggatccaacaggttgaaactaacccagggccaagctgaactttcagggcagttctcaaaataactgagctttgtggtcgttcccgcgcacataccccacaattggaatgatacagagaggatcagcatggcccctgcgcaaggatgacacacaaattcatgaagcagtccgtattttaattttaaaaaaaatgagtttcacaggctgagcgtgtgtggccagagccggactagaaccagatcttctggggcaaaggggcgaggggaatgaatgacatttatccaacacctagttgttttttccctttagcaggaagtcattatgcaacttacacatattcatcagatttcctctgacttacccggacatgtacatgggaatgatgtgcactgccaagaaatgtgggattaggtttcagcctccagctattatcttaatctatgagagtgaaatcaaggggaaaattcgccagcgcattatgccagttcgaaacttttcaaagttttcaggtacctcatgtcttatcttgcctcctgtcttaaatattctctaaatacaggttcacatcatgactctgtcagggactggatgtgtgacactggatgaattacttactctcagtgagcttcagtttcctaatctataaaagggaattgtagtgtttttttccaatctgataattaataatccttttgggattgctgcctaggttaaatgatatagcattattcaataatattaattcccttctctcattccaaatgttttttatttgtagtcttaatattttattaataccactactgctatctaaaagctataatatcacttttcttctcagacatattctccagttgaaagtgtttaatatctctacaaagtgatttttaagttaaagaagtcaaaactgtatctgtccttctcccaccacactgaaagctcaatataaaggaatggttctacaaagtaattcattccaatcaagccatttagctacttgactataatggagataatattttcagggttcagagtttttgttctgttttggctgttgcagtagtttatggcgtatatgatgtacgtggtacgtatgtggtatatagttttcttctctctcccagattgcaccagagctgctgaacaattaaagaataatccgcgacacaagagttacctagaacaagtatccctgaggcagctagagaagctattcagttttttacgaggttacttgtcggggcagagtctggcagaaacaatggaacaaattcaacgggaaacaaccattgatcctgaggaagacctgaacaaactagatgacaaggagcttgccaaaagaaagagcatcatggatgaactttttgagaaaaatcagaagaagaaggatgatccaaattttgtttatgacattgaggttgaatttccacaggacgatcaactgcagtcctgtggctgggacacagagtcagctgatgagttctgataccaaacactcaaaacatgcattgggctagcagaatatccatgtttattaccagactggttctggaagaagctgtaaagaatactaaatatgttgggttataggggattgaccatgttacttttcaaaaccaggacatttaaagcatctactatgtaggtgcatgaggagtatgggaaaaacagaataaaggaatctgcctttaaggagcttacaatcatgccgggtgcggtggctcacgcctgtaatcccagcactttgggaggctgaggcgggtggatcacctaaggtcaggagttcgagaccagcctagccaacatggtgaaacctcgcctctactaaaaatacaaaaattagccaggcgtggtggcgggtgcctgtaatcccggctactcaggaggctgaggcaggagattcgcttgaacctgggaggctgaggttgcagtgagccgagatcgcgccattgtactccagcctgggcgatgagcaaaactccatctcaaaaaaaaaaaaaaaaaaaagcttacaatctgactggaagatgtcaaaacctgtgaaaagctaattagcagtattaagcaacacaaacattagtgccaaatgcatgataaaggctaaagaaggccagagcatatattactgtagagtagaatagtaagggaagactttgtcctttagtaaagagataggaggtggcctggcccttgaaatagtagtgtttaggtagatgcttgtgtaggattcctgataagagcaactgaaaagaaggagaggggaagtagtaaagggacaagaaacaattttttttttgaggaaccataagcaaattatagtttgacaagacaagattgggggacatatatggttaccagggaattacctcttatgtgttatatctttatattatttatctctggaaaagagtaccctgcaaaattccctacagctgcaagcagatgtcacttgatggacagagggggaattctgcccctccggtatcgggaaatacatactaaagacattgcgaaacgctgaacctcttcccataaataaaaggtttgtttgtaaaatgggaaatccacccataataaatgaacaataggcactgccagtttaggcctgttcatgaatggatctgcaagacagcatcttcgtttaacaacattatctgtgatttgatacatttatccttattacaatattgtttagttggtagaaattctatgttttctacaaggaaattgatgtttattaaataaaactgaaaataattactcagtgtttctacctgttcacttccactctccttaccattaagcacttcattaaaagggagctcattcttgtagtgatatcaccagagttttgaaatagtcagatctcatacatacagtagccaaagggagccaaataattttctgaattagagatctttccatatctacatagtattcctttattctttattgtatagatataggtagtttattttttatttgagac

SEQ ID NO: 4 - DNA, 20 bp, Artificial Sequence
  MO-1 Primer
cttgttaggaagccccacag

SEQ ID NO: 5 - DNA, 20 bp, Artificial Sequence
  MO-1 Primer
cgcaaggatgacacacaaat

SEQ ID NO: 6 - DNA, 24 bp, Artificial Sequence
  MO-1 Primer
ggaaactgaagctcactgagagta

SEQ ID NO: 7 - DNA, 25 bp, Artificial Sequence
  MO-1 Primer
aatattttcagggttcagagttttt

SEQ ID NO: 8 - DNA, 23 bp, Artificial Sequence
  MO-1 Primer
ttgaaaagtaacatggtcaatcc

SEQ ID NO: 9 - DNA, 23 bp, Artificial Sequence
  MO-1 Primer
ggattgaccatgttacttttcaa

SEQ ID NO: 10 - protein, 14 aa, Artificial Sequence
  MO-1
CTRAAEQLKNNPRH

SEQ ID NO: 11 - protein, 21 aa, Artificial Sequence
  MO-1
DPNFVYDIEVEFPQDDQLQSC

SEQ ID NO: 12 - DNA, 28 bp, Artificial Sequence
  MO-1 Primer
ttcatcagatttcctctgacttacccgg

SEQ ID NO: 13 - DNA, 29 bp, Artificial Sequence
  MO-1 Primer
gagtgtttggtatgagaactcatcagctg

SEQ ID NO: 14 - DNA, 29 bp, Artificial Sequence
  MO-1 Primer
gtaacatggtcaatcccctataacccaac

SEQ ID NO: 15 - DNA, 24 bp, Artificial Sequence
  MO-1 Primer
ctgccccgacaagtaacctcgtaa

SEQ ID NO: 16 - DNA, 27 bp, Artificial Sequence
  MO-1 Primer
ctgaatagcttctctagctgcctcagg

SEQ ID NO: 17 - DNA, 23 bp, Artificial Sequence
  MO-1 Primer
gggattaggtttcagcctccagc

SEQ ID NO: 18 - DNA, 26 bp, Artificial Sequence
  MO-1 Primer
gaatagcttctctagctgcctcaggg

SEQ ID NO: 19 - DNA, 26 bp, Artificial Sequence
  MO-1 Primer
gaaatcaaggggaaaattcgccagcg

SEQ ID NO: 20 - DNA, 21 bp, Artificial Sequence
  MO-1 Primer
gtttctgccagactctgcccc

SEQ ID NO: 21 - DNA, 26 bp, Artificial Sequence
  MO-1 Primer
gcactgccaagaaatgtgggattagg

SEQ ID NO: 22 - DNA, 26 bp, Artificial Sequence
  MO-1 Primer
ctttaattgttcagcagctctggtgc

SEQ ID NO: 23 - DNA, 22 bp, Artificial Sequence
  MO-1 Primer
gagttaggttccagcctccagc

SEQ ID NO: 24 - DNA, 22 bp, Artificial Sequence
  MO-1 Primer
caggtaactcttgtgccgtggg

SEQ ID NO: 25 - DNA, 25 bp, Artificial Sequence
  MO-1 Primer
gaatgaaaccgaaggaaagagccgc

SEQ ID NO: 26 - DNA, 27 bp, Artificial Sequence
  MO-1 Primer
cgcaaaaaaacaaacagcttctccagc

SEQ ID NO: 27 - DNA, 33 bp, Artificial Sequence
  MO-1 Primer
ttgcccttgaattcagatttcctctgacttacc

SEQ ID NO: 28 - DNA, 28 bp, Artificial Sequence
  MO-1 Primer
ggctgctcgacgggtttaaactcaatgg

SEQ ID NO: 29 - DNA, 27 bp, Artificial Sequence
  MO-1 Primer
ctctaaatacaggttcacatcatgact

SEQ ID NO: 30 - DNA, 25 bp, Artificial Sequence
  MO-1 Primer
gtgtcacacatccagtccctgacag

SEQ ID NO: 31 - DNA, 68 bp, Artificial Sequence
  MO-1 Primer
gatccccaagaataatccgcgacacattttcaagagaaatgtgtcgcggattattctttttttggaaa

SEQ ID NO: 32 - DNA, 68 bp, Artificial Sequence
  MO-1 Primer
agcttttccaaaaaaagaataatccgcgacacatttctcttgaaaatgtgtcgcggattattcttggg

(19) United States
(12) Patent Application Publication
(10) Pub. No.: US 2010/0077496 A1
(43) Pub. Date: Mar. 25, 2010

(54) MO-1, A Gene Associated With Morbid Obesity

(75) Inventors: Adel Shalata, Sakhnin (IL); JOHN Martignetti, Chappaqua, NY (US); Robert Desnick, New York, NY (US)

Correspondence Address: JONES DAY, 222 EAST 41ST ST, NEW YORK, NY, 10017, US

(73) Assignee: Mt. Sinai School of Medicine, New York, NY (US)

(21) Appl. No.: 12/596,776

(22) PCT Filed: Apr. 21, 2008
(86) PCT No.: PCT/US08/05083

§ 371 Date: Oct. 20, 2009

Related U.S. Application Data
(60) Provisional application No. 60/925,401, filed on Apr. 20, 2007.

(51) Int. Cl. A61K 38/16; C07H 21/04; C07K 14/00; C12N 15/63; C12N 5/10; C07K 16/00; A01K 67/027; C12Q 1/68; A61K 31/7088

(52) U.S. Cl. 800/16; 536/23.1; 530/350; 435/320.1; 435/366; 435/370; 530/387.9; 530/387.3; 800/13; 800/14; 800/18; 435/6; 514/12; 514/44 R

(57) ABSTRACT

MO-1 is a newly identified gene and gene product associated with morbid obesity. Isolated MO-1 nucleic acids, MO-1 polypeptides, oligonucleotides that hybridize to MO-1 nucleic adds, and vectors, including expression vectors, comprising MO-1 nucleic acids are disclosed, as are isolated host cells, antibodies, transgenic non-human animals, compositions, and kits relating to MO-1. Methods of detecting the presence of MO-1 nucleic acid, screening for agents which affect MO-1 activity, and screening for MO-1 variants are also disclosed.

### 1. FIELD OF THE INVENTION

The present invention relates to MO-1, a newly identified gene and gene product associated with morbid obesity. In certain aspects, the present invention provides isolated MO-1 nucleic acids, MO-1 polypeptides, oligonucleotides that hybridize to MO-1 nucleic acids, and vectors, including expression vectors, comprising MO-1 nucleic acids. The present invention further provides isolated host cells, antibodies, transgenic non-human animals, compositions, and kits relating to MO-1. In other aspects, the present invention further provides methods of methods of detecting the presence of MO-1 nucleic acid, methods of screening for agents which affect MO-1 activity, and methods of screening for MO-1 variants.

### 2. BACKGROUND OF THE INVENTION

Obesity is a major risk factor for type II diabetes mellitus, heart disease, hypertension, the metabolic syndrome, and cancer and is increasingly prevalent in Western society and in developing countries. See Kopelman P G. Obesity as a medical problem. Nature. 2000 Apr. 6; 404 (6778):635-43. Today, more than 1.1 billion individuals are overweight and more than 300 million are obese. See Hossain P, Kawar B, El Nahas M. Obesity and diabetes in the developing world—a growing challenge. N Engl J Med. 2007 Jan. 18; 356 (3):213-5. Obesity is assessed by the calculation of the body mass index (BMI) [weight/(height)² in kg/m²]. Individuals with a BMI>=30 kg/m² are considered obese, whereas those with a BMI >40 are morbidly obese. Despite intense scrutiny of this worldwide public health problem, the molecular and regulatory mechanisms which underlie the differences between lean and obese individuals remain largely unknown. Obtaining a better understanding of how energy balance is controlled should provide the framework for future clinical intervention and rational drug design.

In humans, the importance of genetic factors in obesity has been clearly defined through numerous twin, familial aggregation, and adoption studies. See Stunkard A J, Sorensen T I, Hanis C, Teasdale T W, Chakraborty R, Schull W J, Schulsinger F. An adoption study of human obesity. N Engl J Med. 1986 Jan. 23; 314 (4):193-8, Stunkard A J, Foch T T, Hrubec Z. A twin study of human obesity. JAMA. 1986 July; 256 (1):51-4, Price R A, Stunkard A J, Ness R, Wadden T, Heshka S, Kanders B, Cormillot A. Childhood onset (age less than 10) obesity has high familial risk. Int J Obes. 1990 February; 14 (2):185-95, and Allison D B, Kaprio J, Korkeila M, Koskenvuo M, Neale M C, Hayakawa K. The heritability of body mass index among an international sample of monozygotic twins reared apart. Int J Obes Relat Metab Disord. 1996 June; 20 (6):501-6. Indeed, through these studies heritability has been estimated as high as 40-90%. See Friedman J M. Modern science versus the stigma of obesity. Nat Med. 2004 June; 10 (6):563-9. In the absence of rational gene candidates, genome-wide genetic association studies have emerged as a potentially powerful tool. And as may be predicted, numerous genome-wide linkage studies have identified novel candidate gene loci for future studies. See Rankinen T, Zuberi A, Chagnon Y C, Weisnagel S J, Argyropoulos G, Walts B, Perusse L, Bouchard C. The human obesity gene map: the 2005 update. Obesity (Silver Spring). 2006 April; 14 (4):529-644. Unfortunately, these linkage studies have generally identified broad chromosomal regions containing scores of candidate genes and ESTs. Two major related problems now exist. First, the large number of genes within these regions need to be individually characterized. Second, biologically plausible gene candidates within these regions are not always intuitively obvious: obesity-related genes may regulate a broad spectrum of physiologic pathways, including those governing satiety, basal metabolic rate, and activity. In addition, novel genes or those unrelated to the present, limited understanding of disease pathophysiology may go undetected.

Most striking with regard to the genetic basis of obesity and providing insights into its molecular basis has been the identification of gene mutations causing a number of Mendelian obesity disorders. See Farooqi S, O'Rahilly S. Genetics of obesity in humans. Endocr Rev. 2006 December; 27 (7):710-18. These include leptin and leptin receptor deficiencies, melanocortin 4 receptor and POMC deficiencies and the pleiotropic syndromes Prader-Willi and Bardet-Biedl. See Bell C G, Walley A J, Froguel P. The genetics of human obesity. Nat Rev Genet. 2005 March; 6 (3):221-34. Unfortunately, while each have provided insight into the molecular basis by which the hypothalamus controls satiety and energy homeostasis, none has provided insight into more common forms of obesity nor has yet provided a useful drug target for obesity and its comorbid features including diabetes. The present invention is intend to address these unmet needs.

### 3. SUMMARY OF THE INVENTION

The present invention is based in part on the discovery of a novel gene, termed MO-1, which exhibits partial structural similarity to phosphoenolpyruvate carboxykinase, mutations of which are associated with morbid obesity. A MO-1 cDNA has been cloned and sequenced, and a MO-1 amino acid sequence has been determined.

Accordingly, in a first aspect, the present invention provides an isolated polypeptide comprising an amino acid sequence having at least 70% identity to a MO-1 amino acid sequence (SEQ ID NO:1). In one embodiment, the isolated polypeptide comprises the amino acid sequence of SEQ ID NO:1.

In another aspect, the invention provides an isolated nucleic acid encoding a polypeptide comprising an amino acid sequence having at least 70% identity to a MO-1 amino acid sequence (SEQ ID NO:1). In certain embodiments, the isolated nucleic acid encodes a polypeptide comprising the amino acid sequence of SEQ ID NO:1. In certain embodiments, the isolated nucleic acid comprises a nucleic acid sequence having at least 70% identity to at least about 500 contiguous nucleotides selected from SEQ ID NO:2 or the complement thereof. In certain embodiments, the isolated nucleic acid comprises at least about 500 nucleotides selected from the nucleic acid sequence of SEQ ID NO:2, or the complement thereof. In a particular embodiment, the isolated nucleic acid comprises the nucleic acid sequence of SEQ ID NO:2, or the complement thereof.

In another aspect, the invention provides an isolated oligonucleotide comprising at least about 10 consecutive nucleotides of SEQ ID NO:2 or its complementary strand.

In another aspect, the invention provides a vector comprising a nucleic acid of the invention. In certain embodiments, the vector comprises at least about 500 nucleotides selected from the nucleic acid sequence of SEQ ID NO:2. In certain embodiments, the vector comprises a nucleic acid that encodes the polypeptide of SEQ ID NO:1. In certain embodiments, the vector comprises the nucleic acid sequence of SEQ ID NO:2. In a particular embodiment, the MO-1 nucleic acid sequence in the vector is operably linked to a transcriptional regulatory sequence. In certain embodiments, the vector is selected from the group comprising a plasmid, a cosmid, a virus, and a bacteriophage. In certain embodiments, the vector expresses a polypeptide comprising SEQ ID NO:1 in a cell transformed with said vector. In certain embodiments, the polypeptide encoded by the vector can be expressed in adipocytes.

In another aspect, the invention provides an isolated host cell comprising a MO-1 nucleic acid according to the present invention. In another aspect, the invention provides an isolated host cell comprising a vector that expresses MO-1. In certain embodiments, the isolated host cell is an adipocyte.

In another aspect, the invention provides an isolated antibody that specifically binds to a polypeptide comprising an amino acid sequence of SEQ ID NO:1. In certain embodiments, the antibody is a polyclonal, monoclonal, single chain monoclonal, recombinant, chimeric, humanized, mammalian, or human antibody.

In another aspect, the invention provides a transgenic non-human animal, which expresses a transgenic nucleic acid encoding MO-1 polypeptide. In certain embodiments, the transgenic non-human animal MO-1 polypeptide comprises the amino acid sequence of SEQ ID NO:1. In a particular embodiment, the transgenic non-human animal over- or under-expresses MO-1 polypeptide relative to wild-type expression of MO-1 polypeptide in a human adipocyte. In one embodiment, the transgenic non-human animal comprises a transgenic nucleic acid having at least 70% identity to SEQ ID NO:2, or the complement thereof. In certain embodiments, the transgenic non-human animal is a mammal, including, but not limited to, a mouse, rat, rabbit, hamster, pig, goat, or sheep.

In another aspect, the invention provides a transgenic non-human animal whose germ cells comprise a homozygous null mutation in the endogenous nucleic acid sequence encoding MO-1. For example, such an animal can be one wherein the mutation is created by insertion of, e.g., a neomycin cassette, in reverse orientation to MO-1 transcription and wherein said mutation has been introduced into said animal by homologous recombination in an embryonic stem cell such that said animal does not express a functional MO-1 polypeptide. In certain embodiments, the transgenic non-human animal is fertile and transmits said null mutation to its offspring. In particular embodiments, the transgenic non-human animal is a mammal, including, but not limited to, a mouse, rat, rabbit, hamster, or sheep. In certain embodiments, the animal exhibits a phenotype associated with mutations of MO-1, e.g., obesity.

In another aspect, the invention provides a method of screening for agents which affect MO-1 activity, comprising: a) administering said agent to a cell that expresses a MO-1 polypeptide; and b) assessing a biological activity of the MO-1 in the cell. In some embodiments, the agent agonizes, e.g., increases, the MO-1 activity. In some embodiments, the agent antagonizes, e.g., decreases, the MO-1 activity.

In another aspect, the invention provides a method of screening for agents which affect MO-1 activity, comprising: a) administering said agent to a transgenic non-human animal according to the present invention; and b) assessing the animal for an alteration in a metabolic function affected by said agent. In certain embodiments, said metabolic function relates to glucose or lipid metabolism.

In another aspect, the invention provides a method of detecting the presence of the MO-1 nucleic acid in a sample, comprising: (a) contacting the sample with a nucleic acid that hybridizes to the MO-1 nucleic acid; and (b) determining whether the nucleic acid binds to a nucleic acid in the sample.

In another aspect, the invention provides a composition comprising a MO-1 polypeptide of the invention and a pharmaceutically acceptable carrier. In another aspect, the invention provides a composition comprising a polypeptide having an amino acid sequence that comprises SEQ ID NO:1 and a pharmaceutically acceptable carrier.

In another aspect, the invention provides a composition comprising a MO-1-encoding nucleic acid of the invention and a pharmaceutically acceptable carrier. In another aspect, the invention provides a composition comprising a polynucleotide encoding a polypeptide having an amino acid sequence that comprises SEQ ID NO:1 and a pharmaceutically acceptable carrier. In certain embodiments, the polynucleotide comprises a nucleotide sequence of SEQ ID NO:2.

In another aspect, the invention provides a kit comprising i) an isolated oligonucleotide comprising at least 10 consecutive nucleotides of SEQ ID NO:2, or its complementary strand; and ii) a container. In certain embodiments, the kit contains the oligonucleotide which comprises at least 15 consecutive nucleotides of SEQ ID NO:2 or its complementary strand.

### 4. BRIEF DESCRIPTION OF THE DRAWINGS

FIG. 1 presents a diagram showing the lineage of a large consanguineous family with a high incidence of mutant MO-1.

FIG. 2 presents a diagram showing a representative vector suitable for use in generating a MO-1 knockout mouse.

FIG. 3 presents an exemplary PCR screen used to identify transgenic mice comprising a MO-1 nucleic acid.

FIG. 4 presents a diagram showing weights of mice overexpressing MO-1.

FIG. 5 presents a diagram showing glucose tolerance of transgenic mice expressing human MO-1.

FIG. 6 presents another diagram showing glucose tolerance of transgenic mice expressing human MO-1.

FIG. 7A presents a diagram showing single nucleotide polymorphisms associated with altered body mass phenotypes.

FIG. 7B presents a table showing associations between single nucleotide polymorphisms and altered body mass phenotypes.

FIG. 8 presents a diagram showing the results of overexpression of MO-1 in Hep3B cells.

FIG. 9 presents a diagram showing the results of inhibiting expression of MO-1 in Hep3B cells.

FIG. 10 presents diagram showing the results of inhibiting expression of MO-1 in NIH 3T3 L1 cells.

FIG. 11 presents the results of analysis of downstream effects on gene expression of inhibiting expression of MO-1 in NIH 3T3 L1 cells.

FIG. 12 presents a diagram showing quantitatively the downstream effects on gene expression of inhibiting expression of MO-1 in NIH 3T3 L1 cells.

FIG. 13 presents a diagram showing expression levels of MO-1 in human tissues.

FIG. 14 presents a diagram showing expression levels of MO-1 in differentiating mouse 3T3 L1 cells.

FIG. 15 presents a diagram showing identification of Insulin-Regulated Membrane Aminopeptidase as a potential binding partner for MO-1.

FIG. 16 presents a diagram showing the effects on proliferation of inhibiting MO-1 expression in Hep3B cells.

### 5. DETAILED DESCRIPTION OF THE INVENTION

This disclosure provides, for the first time, an isolated cDNA molecule which, when transfected into cells can produce MO-1 protein. MO-1 protein is believed to be linked to, inter alia, energy metabolism, e.g., glucose or lipid metabolism. This disclosure provides the molecule, the nucleotide sequence of this cDNA and the amino acid sequence of MO-1 protein encoded by this cDNA.

Having herein provided the nucleotide sequence of the MO-1 cDNA, correspondingly provided are the complementary DNA strands of the cDNA molecule, and DNA molecules which hybridize under stringent conditions to MO-1 cDNA molecule, or its complementary strand. Such hybridizing molecules include DNA molecules differing only by minor sequence changes, including nucleotide substitutions, deletions and additions. Also comprehended by this invention are isolated oligonucleotides comprising at least a portion of the cDNA molecule or its complementary strand. These oligonucleotides can be employed as effective DNA hybridization probes or primers for use in the polymerase chain reaction. Such probes and primers may be particularly useful in the screening and diagnosis of persons genetically predisposed to obesity and other forms of metabolic dysfunction, as the result of MO-1 gene mutations.

Recombinant DNA vectors comprising the disclosed DNA molecules, and transgenic host cells containing such recombinant vectors, are also provided. Disclosed embodiments also include transgenic nonhuman animals which over-or under-express MO-1 protein, or over-or under-express fragments or variants of MO-1 protein.

For clarity of disclosure, and not by way of limitation, the detailed description of the invention hereinafter is divided into the subsections that follow. All publications mentioned herein are incorporated by reference to disclose and describe the methods and/or materials in connection with which the publications are cited.

5.1 Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an,” and “the” mean “at least one” or “one or more” unless the context clearly dictates otherwise.

As used herein, “MO-1” refers to proteins or peptides which have an amino acid sequence that is identical to SEQ ID NO:1, as well as proteins sharing sequence similarity, e.g., 70%, 75%, 80%, 85%, 90%, 95%, or greater percent identity, with the amino acid sequence of SEQ ID NO:1. Further, these proteins have a biological activity in common with the polypeptide having the amino acid sequence of SEQ ID NO:1, including, but not limited to, antigenic cross-reactivity, autoinhibition, phosphorylation activity, and the like. It is also contemplated that a MO-1 protein can have one or more conservative or non-conservative amino acid substitutions, or additions or deletions from the amino acid sequence of SEQ ID NO:1 so long as the protein having such sequence alteration shares a biological activity as described above with the polypeptide of SEQ ID NO:1. MO-1 also includes proteins or peptides expressed from different mutations, different spliced forms and various sequence polymorphisms of the MO-1 gene.

As used herein, “functional fragments and variants of MO-1” refer to those fragments and variants that maintain one or more functions of MO-1. It is recognized that the gene or cDNA encoding MO-1 can be considerably mutated without materially altering one or more MO-1 functions. First, the genetic code is well-known to be degenerate, and thus different codons may encode the same amino acids. Second, even where an amino acid substitution is introduced, the mutation can be conservative and have no material impact on the essential functions of MO-1. Third, part of the MO-1 polypeptide can be deleted without impairing or eliminating all of its functions. Fourth, insertions or additions can be made in MO-1, for example, adding epitope tags, without impairing or eliminating its functions. Other modifications can be made without materially impairing one or more functions of MO-1, for example, in vivo or in vitro chemical and biochemical modifications which incorporate unusual amino acids. Such modifications include, for example, acetylation, carboxylation, phosphorylation, glycosylation, ubiquination, labeling with radionuclides, and various enzymatic modifications, as will be readily appreciated by those skilled in the art. A variety of methods for labeling proteins and substituents or labels useful for such purposes are well known in the art, and include radioactive isotopes such as ligands which bind to labeled antiligands (e.g., antibodies), fluorophores, chemiluminescent agents, enzymes, and antiligands. Functional fragments and variants can be of varying length. For example, some fragments have at least 10, 25, 50, 75, 100, or 200 or more amino acid residues.

As used herein, “protein” is synonymous with “polypeptide” or “peptide” unless the context clearly dictates otherwise.

As used herein, a “MO-1 gene” refers to a gene that encodes MO-1 as defined herein. A mutation of MO-1 gene includes nucleotide sequence changes, additions or deletions, including deletion of large portions or the entire MO-1 gene, or duplications of all or substantially all of the gene. Alternatively, genetic expression of MO-1 can be deregulated such that MO-1 is over or under expressed. The term “MO-1 gene” is understood to include the various sequence polymorphisms and allelic variations that exist within the population. This term relates primarily to an isolated coding sequence, but can also include some or all of the flanking regulatory elements and/or intron sequences. The RNA transcribed from a mutant MO-1 gene is mutant MO-1 messenger RNA.

As used herein, “MO-1 cDNA” refers to a cDNA molecule which, when transfected or otherwise introduced into cells, expresses the MO-1 protein. The MO-1 cDNA can be derived, for instance, by reverse transcription from the mRNA encoded by the MO-1 gene and lacks internal non-coding segments and transcription regulatory sequences present in the MO-1 gene. The prototypical human MO-1 cDNA is shown as SEQ ID NO:2.

As used herein, “vector” refers to discrete elements that are used to introduce heterologous DNA into cells for either expression or replication thereof. Selection and use of such vehicles are well known within the skill of the artisan. An expression vector includes vectors capable of expressing DNA that are operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “transgenic animals” refers to non-human animals, preferably mammals, more preferably rodents such as rats or mice, in which one or more of the cells includes a transgene. Other transgenic animals include primates, sheep, rabbits, hamsters, dogs, cows, goats, chickens, amphibians, etc. A “transgene” is exogenous DNA that is integrated into the genome of a cell from which a transgenic animal develops, and which remains in the genome of the mature animal. A “transgene” is intended to encompass exogenous DNA that comprises the coding sequence of a polypeptide, e.g., a MO-1 polypeptide, as well as exogenous DNA that comprises regulatory sequences, e.g., promoted or enhancer sequences, that affect expression levels of an endogenous polypeptide, e.g., a MO-1 polypeptide.

As used herein, a “homologous recombinant animal” refers to a non-human animal, preferably a mammal, more preferably a rodent such as a rat or mouse, in which the endogenous MO-1 gene has been altered by an exogenous DNA molecule that recombines homologously with endogenous MO-1 in a (e.g., embryonic) cell prior to development of the animal. Other homologous recombinant animals include rabbits, hamsters and sheep. Host cells with exogenous MO-1 can be used to produce non-human transgenic animals, such as fertilized oocytes or embryonic stem cells into which MO-1-encoding sequences have been introduced. Such host cells can then be used to create non-human transgenic animals or homologous recombinant animals.

As used herein, the term “biological sample” includes tissues, cells and biological fluids isolated from a subject, as well as tissues, cells and fluids present within a subject.

As used herein, the term “pharmaceutically acceptable” means approved by a regulatory agency of the Federal or a state government, or listed in the U.S. Pharmacopeia or other generally recognized pharmacopeia for use in animals, and more particularly in humans.

As used herein, the term “carrier” refers to a diluent, adjuvant, excipient, or vehicle with which a therapeutic of the invention is administered. Such pharmaceutical carriers can be sterile liquids, such as water and oils, including those of petroleum, animal, vegetable or synthetic origin, such as peanut oil, soybean oil, mineral oil, sesame oil and the like.

As used herein, an “effective amount” of an active agent for treating a particular disease is an amount that is sufficient to ameliorate, or in some manner reduce the symptoms associated with the disease. The amount may cure the disease but, typically, is administered in order to ameliorate the symptoms of the disease.

As used herein, “active agent” means any substance intended for the diagnosis, cure, mitigation, treatment, or prevention of disease in humans and other animals, or to otherwise enhance physical and mental well being.

The terms “treatment,” “treating,” and the like are used herein to generally mean obtaining a desired pharmacological and/or physiological effect in a subject actively suffering from a condition. The effect may completely or partially treat a disease or symptom thereof and thus may be therapeutic in terms of a partial or complete cure for a disease and/or adverse effect attributable to the disease. “Treatment” as used herein covers any treatment of a disease in a mammal, particularly a human, and includes inhibiting the disease, i.e., arresting its development; or relieving the disease, i.e., causing regression of the disease. In one example, treatment refers to treating patients with, or at risk for, development of obesity and related conditions. More specifically, “treatment” is intended to mean providing a therapeutically detectable and beneficial effect on a patient suffering from a metabolic disorder.

The terms “prevent,” “preventing,” and the like are used herein to generally refer to preventing a disease from occurring in a subject which may be predisposed to the disease but has not yet been diagnosed as suffering from the disease. Thus, “prevent” can refer to prophylactic or preventative measures, wherein the object is to prevent or slow down (lessen) obesity or onset of obesity.

The term “about,” as used herein, unless otherwise indicated, refers to a value that is no more than 10% above or below the value being modified by the term. For example, the term “about 5 μg/kg” means a range of from 4.5 μg/kg to 5.5 μg/kg. As another example, “about 1 hour” means a range of from 48 minutes to 72 minutes.

5.2 Polypeptides of the Invention

The present invention provides newly identified and isolated polypeptides referred to in the present application as MO-1. In some embodiments, the polypeptides are native sequence MO-1 polypeptides. In some embodiments, the polypeptides comprise substantially the same amino acid sequences as found in the native MO-1 sequences. In certain embodiments, the invention provides amino acid sequences of functional fragments and variants of MO-1 that comprise an antigenic determinant (i.e., a portion of a polypeptide that can be recognized by an antibody) or which are otherwise functionally active, as well as nucleic acids encoding the foregoing. MO-1 functional activity encompasses one or more known functional activities associated with a full-length (wild-type) MO-1 polypeptide, e.g., antigenicity (the ability to be bound by an antibody to a protein consisting of the amino acid sequence of SEQ ID NO: 1); immunogenicity (the ability to induce the production of an antibody that binds SEQ ID NO: 1), and so forth.

In some embodiments, the polypeptides comprise the amino acid sequences having functionally inconsequential amino acid substitutions, and thus have amino acid sequences which differ from that of the native MO-1 sequence. Substitutions can be introduced by mutation into MO-1-encoding nucleic acid sequences that result in alterations in the amino acid sequences of the encoded MO-1 but do not alter MO-1 function. For example, nucleotide substitutions leading to amino acid substitutions at “non-essential” amino acid residues can be made in MO-1 encoding sequences. A “non-essential” amino acid residue is a residue that can be altered from the wild-type sequence of MO-1 without altering biological activity, whereas an “essential” amino acid residue is required for such biological activity. For example, amino acid residues that are conserved among MO-1 polypeptides are predicted to be particularly unsuitable for alteration. Amino acids for which conservative substitutions can be made are well known in the art.

Useful conservative substitutions are shown in Table 1, “Preferred Substitutions.” Conservative substitutions whereby an amino acid of one class is replaced with another amino acid of the same type fall within the scope of the subject invention so long as the substitution does not materially alter the biological activity of the compound. If such substitutions result in a change in biological activity, then more substantial changes, indicated in Table 2 as exemplary are introduced and the products screened for MO-1 polypeptide biological activity.

TABLE 1
Preferred Substitutions

| | | |
|---|---|---|
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, Norleucine | Leu |

Non-conservative substitutions that effect: (1) the structure of the polypeptide backbone, such as a β-sheet or α-helical conformation; (2) the charge; (3) hydrophobicity; or (4) the bulk of the side chain of the target site, can modify MO-1 polypeptide function or immunological identity. Residues are divided into groups based on common side-chain properties as denoted in Table 2. Non-conservative substitutions entail exchanging a member of one of these classes for another class. Substitutions may be introduced into conservative substitution sites or more preferably into non-conserved sites.

TABLE 2
Amino acid classes

| Class | Amino Acids |
|---|---|
| hydrophobic | Norleucine, Met, Ala, Val, Leu, Ile |
| neutral hydrophilic | Cys, Ser, Thr |
| acidic | Asp, Glu |
| basic | Asn, Gln, His, Lys, Arg |
| disrupt chain conformation | Gly, Pro |
| aromatic | Trp, Tyr, Phe |

The variant polypeptides can be made using methods known in the art such as oligonucleotide-mediated (site-directed) mutagenesis, alanine scanning, and PCR mutagenesis. Site-directed mutagenesis (see Carter, Biochem. J. 237:1-7 (1986); Zoller and Smith, Methods Enzymol. 154:329-50 (1987)), cassette mutagenesis, restriction selection mutagenesis (Wells et al., Gene 34:315-323 (1985)) or other known techniques can be performed on cloned MO-1-encoding DNA to produce MO-1 variant DNA (Ausubel et al., Current Protocols In Molecular Biology, John Wiley and Sons, New York (current edition); Sambrook et al., Molecular Cloning, A Laboratory Manual, 3d. ed., Cold Spring Harbor Laboratory Press, Cold Spring Harbor, N.Y. (2001).

In certain embodiments, MO-1 used in the present invention includes MO-1 mutants or derivatives having an amino acid substitution with a non-classical amino acid or chemical amino acid analog. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

In one embodiment, the present invention includes an isolated polypeptide comprising an amino acid sequence having at least 70% identity to SEQ ID NO:1. In some embodiments, the polypeptide comprises an amino acid sequence having at least 75%, 80%, 85%, 90%, or 95% identity to SEQ ID NO:1. In a particular embodiment, the isolated polypeptide comprises the amino acid sequence of SEQ ID NO:1.

Percent identity in this context means the percentage of amino acid residues in the candidate sequence that are identical (i.e., the amino acid residues at a given position in the alignment are the same residue) or similar (i.e., the amino acid substitution at a given position in the alignment is a conservative substitution, as discussed above), to the corresponding amino acid residue in the peptide after aligning the sequences and introducing gaps, if necessary, to achieve the maximum percent sequence homology. In certain embodiments, a MO-1 homologue is characterized by its percent sequence identity or percent sequence similarity with the naturally occurring MO-1 sequence. Sequence homology, including percentages of sequence identity and similarity, are determined using sequence alignment techniques well-known in the art, preferably computer algorithms designed for this purpose, using the default parameters of said computer algorithms or the software packages containing them.

Non-limiting examples of computer algorithms and software packages incorporating such algorithms include the following. The BLAST family of programs exemplify a preferred, non-limiting example of a mathematical algorithm utilized for the comparison of two sequences (e.g., Karlin & Altschul, 1990, Proc. Natl. Acad. Sci. USA 87:2264-2268 (modified as in Karlin & Altschul, 1993, Proc. Natl. Acad. Sci. USA 90:5873-5877), Altschul et al., 1990, J. Mol. Biol. 215:403-410, (describing NBLAST and XBLAST), Altschul et al., 1997, Nucleic Acids Res. 25:3389-3402 (describing Gapped BLAST, and PSI-Blast). Another preferred example is the algorithm of Myers and Miller (1988 CABIOS 4:11-17) which is incorporated into the ALIGN program (version 2.0) and is available as part of the GCG sequence alignment software package. Also preferred is the FASTA program (Pearson W. R. and Lipman D. J., Proc. Nat. Acad. Sci. USA, 85:2444-2448, 1988), available as part of the Wisconsin Sequence Analysis Package. Additional examples include BESTFIT, which uses the “local homology” algorithm of Smith and Waterman (Advances in Applied Mathematics, 2:482-489, 1981) to find best single region of similarity between two sequences, and which is preferable where the two sequences being compared are dissimilar in length; and GAP, which aligns two sequences by finding a “maximum similarity” according to the algorithm of Neddleman and Wunsch (J. Mol. Biol. 48:443-354, 1970), and is preferable where the two sequences are approximately the same length and an alignment is expected over the entire length.

Examples of homologues may be the ortholog proteins of other species including animals, plants, yeast, bacteria, and the like. Homologues may also be selected by, e.g., mutagenesis in a native protein. For example, homologues may be identified by site-specific mutagenesis in combination with assays for detecting protein-protein interactions. Additional methods, e.g., protein affinity chromatography, affinity blotting, in vitro binding assays, and the like, will be apparent to skilled artisans apprised of the present invention.

For the purpose of comparing two different nucleic acid or polypeptide sequences, one sequence (test sequence) may be described to be a specific “percent identical to” another sequence (reference sequence) in the present disclosure. In this respect, when the length of the test sequence is less than 90% of the length of the reference sequence, the percentage identity is determined by the algorithm of Myers and Miller, Bull. Math. Biol., 51:5-37 (1989) and Myers and Miller, Comput. Appl. Biosci., 4 (1):11-17 (1988). Specifically, the identity is determined by the ALIGN program. The default parameters can be used.

Where the length of the test sequence is at least 90% of the length of the reference sequence, the percentage identity is determined by the algorithm of Karlin and Altschul, Proc. Natl. Acad. Sci. USA, 90:5873-77 (1993), which is incorporated into various BLAST programs. Specifically, the percentage identity is determined by the “BLAST 2 Sequences” tool. See Tatusova and Madden, FEMS Microbiol. Lett., 174 (2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter).

5.3 Nucleic Acids of the Invention

In another aspect, the present invention provides newly identified and isolated nucleotide sequences encoding MO-1. In particular, nucleic acids encoding native sequence human MO-1 polypeptides have been identified and isolated.

The MO-1-encoding or related sequences provided by the instant invention include those nucleotide sequences encoding substantially the same amino acid sequences as found in native MO-1, as well as those encoded amino acid sequences having functionally inconsequential amino acid substitutions, and thus having amino acid sequences which differ from that of the native sequence. Examples include the substitution of one basic residue for another (i.e. Arg for Lys), the substitution of one hydrophobic residue for another (i.e. Leu for Ile), or the substitution of one aromatic residue for another (i.e. Phe for Tyr, etc.).

The invention further relates to fragments of MO-1. Nucleic acids encoding such fragments are thus also within the scope of the invention. The MO-1 gene and MO-1-encoding nucleic acid sequences of the invention include human and related genes (homologues) in other species. In some embodiments, the MO-1 gene and MO-1-encoding nucleic acid sequences are from vertebrates, or more particularly, mammals. In a preferred embodiment of the invention, the MO-1 gene and MO-1-encoding nucleic acid sequences are of human origin.

In one aspect, the invention provides an isolated nucleic acid encoding a polypeptide comprising an amino acid sequence having at least 70% identity to SEQ ID NO:1. In some embodiments, the nucleic acid encodes a polypeptide comprising an amino acid sequence having at least 75%, 80%, 85%, 90%, or 95% identity to SEQ ID NO:1. In a particular embodiment, the isolated nucleic acid encodes a polypeptide comprising the amino acid sequence of SEQ ID NO:1.

In another embodiment, the invention provides an isolated nucleic acid comprising a nucleic acid sequence having at least 70% identity to at least about 500 contiguous nucleotides selected from SEQ ID NO:2 or the complement thereof. In some embodiments, the nucleic acid comprises a nucleic acid sequence having at least 70% identity to at least about 500, 600, 700, 800, 900, 1000, 1100, 1200, or 1400 contiguous nucleotides selected from SEQ ID NO:2. In some embodiments, the nucleic acid comprises a nucleic acid sequence having at least 75% identity to at least about 500 contiguous nucleotides selected from SEQ ID NO:2. In some embodiments, the nucleic acid comprises a nucleic acid sequence having at least 75% identity to at least about 500, 600, 700, 800, 900, 1000, 1100, 1200, or 1400 contiguous nucleotides selected from SEQ ID NO:2. In some embodiments, the nucleic acid comprises a nucleic acid sequence having at least 80% identity to at least about 500 contiguous nucleotides selected from SEQ ID NO:2. In some embodiments, the nucleic acid comprises a nucleic acid sequence having at least 80% identity to at least about 500, 600, 700, 800, 900, 1000, 1100, 1200, or 1400 contiguous nucleotides selected from SEQ ID NO:2. In some embodiments, the nucleic acid comprises a nucleic acid sequence having at least 85% identity to at least about 500 contiguous nucleotides selected from SEQ ID NO:2. In some embodiments, the nucleic acid comprises a nucleic acid sequence having at least 85% identity to at least about 500, 600, 700, 800, 900, 1000, 1100, 1200, or 1400 contiguous nucleotides selected from SEQ ID NO:2. In some embodiments, the nucleic acid comprises a nucleic acid sequence having at least 90% identity to at least about 500 contiguous nucleotides selected from SEQ ID NO:2. In some embodiments, the nucleic acid comprises a nucleic acid sequence having at least 90% identity to at least about 500, 600, 700, 800, 900, 1000, 1100, 1200, or 1400 contiguous nucleotides selected from SEQ ID NO:2. In some embodiments, the nucleic acid comprises a nucleic acid sequence having at least 95% identity to at least about 500 contiguous nucleotides selected from SEQ ID NO:2. In some embodiments, the nucleic acid comprises a nucleic acid sequence having at least 95% identity to at least about 500, 600, 700, 800, 900, 1000, 1100, 1200, or 1400 contiguous nucleotides selected from SEQ ID NO:2. In certain embodiments, the isolated nucleic acid comprises at least about 500 nucleotides selected from the nucleic acid sequence of SEQ ID NO:2, or the complement thereof. In certain embodiments, the isolated nucleic acid comprises at least about 500, 600, 700, 800, 900, 1000, 1100, 1200, or 1400 nucleotides selected from the nucleic acid sequence of SEQ ID NO:2, or the complement thereof. In a particular embodiment, the isolated nucleic acid comprises the nucleic acid sequence of SEQ ID NO:2, or the complement thereof.

In another aspect, the invention provides an isolated nucleic acid comprising a nucleic acid sequence having at least 70% identity to at least about 500 contiguous nucleotides selected from SEQ ID NO:3 or the complement thereof SEQ ID NO:3 presents the genomic sequence of a human MO-1 gene, including introns and exons. In some embodiments, the nucleic acid comprises a nucleic acid sequence having at least 70% identity to at least about 500, 600, 700, 800, 900, 1000, 1100, 1200, 1500, 2000, or 2500 contiguous nucleotides selected from SEQ ID NO:3. In some embodiments, the nucleic acid comprises a nucleic acid sequence having at least 75% identity to at least about 500 contiguous nucleotides selected from SEQ ID NO:3. In some embodiments, the nucleic acid comprises a nucleic acid sequence having at least 75% identity to at least about 500, 600, 700, 800, 900, 1000, 1100, 1200, 1500, 2000, or 2500 contiguous nucleotides selected from SEQ ID NO:3. In some embodiments, the nucleic acid comprises a nucleic acid sequence having at least 80% identity to at least about 500 contiguous nucleotides selected from SEQ ID NO:3. In some embodiments, the nucleic acid comprises a nucleic acid sequence having at least 80% identity to at least about 500, 600, 700, 800, 900, 1000, 1100, 1200, 1500, 2000, or 2500 contiguous nucleotides selected from SEQ ID NO:3. In some embodiments, the nucleic acid comprises a nucleic acid sequence having at least 85% identity to at least about 500 contiguous nucleotides selected from SEQ ID NO:3. In some embodiments, the nucleic acid comprises a nucleic acid sequence having at least 85% identity to at least about 500, 600, 700, 800, 900, 1000, 1100, 1200, 1500, 2000, or 2500 contiguous nucleotides selected from SEQ ID NO:3. In some embodiments, the nucleic acid comprises a nucleic acid sequence having at least 90% identity to at least about 500 contiguous nucleotides selected from SEQ ID NO:3. In some embodiments, the nucleic acid comprises a nucleic acid sequence having at least 90% identity to at least about 500, 600, 700, 800, 900, 1000, 1100, 1200, 1500, 2000, or 2500 contiguous nucleotides selected from SEQ ID NO:3. In some embodiments, the nucleic acid comprises a nucleic acid sequence having at least 95% identity to at least about 500 contiguous nucleotides selected from SEQ ID NO:3. In some embodiments, the nucleic acid comprises a nucleic acid sequence having at least 95% identity to at least about 500, 600, 700, 800, 900, 1000, 1100, 1200, 1500, 2000, or 2500 contiguous nucleotides selected from SEQ ID NO:3. In certain embodiments, the isolated nucleic acid comprises at least about 500 nucleotides selected from the nucleic acid sequence of SEQ ID NO:3, or the complement thereof. In certain embodiments, the isolated nucleic acid comprises at least about 500, 600, 700, 800, 900, 1000, 1100, 1200, 1500, 2000, or 2500 nucleotides selected from the nucleic acid sequence of SEQ ID NO:3, or the complement thereof. In a particular embodiment, the isolated nucleic acid comprises the nucleic acid sequence of SEQ ID NO:3, or the complement thereof. In certain embodiments, the nucleic acid is not a member of a nucleic acid library. In certain embodiments, the nucleic acid is not a member of a genomic library. In certain embodiments, the nucleic acid is not a member of an expression library.

The present invention also includes nucleic acids that hybridize to or are complementary to the foregoing sequences. In specific aspects, nucleic acids are provided which comprise a sequence complementary to at least 20, 30, 40, 50, 100, 200 nucleotides or the entire coding region of MO-1, or the reverse complement (antisense) of any of these sequences. In a specific embodiment, a nucleic acid which hybridizes to a MO-1 nucleic acid sequence (e.g., having part or the whole of sequence SEQ ID NO:2, or the complements thereof), under conditions of low stringency is provided.

By way of example and not limitation, procedures using such conditions of low stringency are as follows (see also Shilo and Weinberg, 1981, Proc. Natl. Acad. Sci. U.S.A. 78:6789-6792). Filters containing DNA can be pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations can be carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe can be used. Filters can be incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution can then be replaced with fresh solution and incubated an additional 1.5 h at 60° C. Filters may be blotted dry and exposed for autoradiography. If necessary, filters may be washed for a third time at 65-68° C. and re-exposed to film. Other conditions of low stringency which may be used are well known in the art (e.g., as employed for cross-species hybridizations).

In another specific embodiment, a nucleic acid that hybridizes to a nucleic acid encoding MO-1, or its reverse complement, under conditions of high stringency is provided. By way of example and not limitation, procedures using such conditions of high stringency are as follows. Prehybridization of filters containing DNA may be carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters may be hybridized for 48 h at 65° C. in prehybridization mixture containing 100 μg/ml denatured salmon sperm DNA and 5-20×10⁶ cpm of ³²P-labeled probe. Washing of filters may be done at 37° C. for 1 h in a solution containing 2×SSC, 0.01% PVP, 0.01% Ficoll, and 0.01% BSA. This can be followed by a wash in 0.1×SSC at 50° C. for 45 minutes before autoradiography. Other conditions of high stringency that may be used are well known in the art.

5.3.1 Cloning of the MO-1 Gene or cDNA

The present invention further provides methods and compositions relating to the cloning of a gene or cDNA encoding MO-1. In one embodiment of the invention, expression cloning (a technique commonly known in the art), may be used to isolate a gene or cDNA encoding MO-1. An expression library may be constructed by any method known in the art. In one embodiment, mRNA (e.g., human) is isolated, and cDNA is made and ligated into an expression vector such that the cDNA is capable of being expressed by the host cell into which it is introduced. Various screening assays can then be used to select for the expressed MO-1 product. In one embodiment, anti-MO-1 antibodies can be used for selection.

In another embodiment of the invention, polymerase chain reaction (PCR) may be used to amplify desired nucleic acid sequences of the present invention from a genomic or cDNA library. Isolated oligonucleotide primers representing known MO-1-encoding sequences can be used as primers in PCR. In certain embodiments, the isolated oligonucleotide primer comprises at least 10 consecutive nucleotides of SEQ ID NO:2 or its complimentary strand. In certain embodiments, the oligonucleotide comprises the nucleic acid sequence of SEQ ID NO:4. In certain embodiments, the oligonucleotide comprises the nucleic acid sequence of SEQ ID NO:5. In certain embodiments, the oligonucleotide comprises the nucleic acid sequence of SEQ ID NO:6. In certain embodiments, the oligonucleotide comprises the nucleic acid sequence of SEQ ID NO:7. In certain embodiments, the oligonucleotide comprises the nucleic acid sequence of SEQ ID NO:8. In certain embodiments, the oligonucleotide comprises the nucleic acid sequence of SEQ ID NO:9. In certain embodiments, the oligonucleotide comprises the nucleic acid sequence of SEQ ID NO:10. The synthetic oligonucleotides may be utilized as primers to amplify by PCR sequences from RNA or DNA, preferably a cDNA library, of potential interest. Alternatively, one can synthesize degenerate primers for use in the PCR reactions.

In the PCR reactions, the nucleic acid being amplified can include RNA or DNA, for example, mRNA, cDNA or genomic DNA from any eukaryotic species. PCR can be carried out, e.g., by use of a Perkin-Elmer Cetus thermal cycler and Taq polymerase. It is also possible to vary the stringency of hybridization conditions used in priming the PCR reactions, to allow for greater or lesser degrees of nucleotide sequence similarity between a known MO-1 nucleotide sequence and a nucleic acid homologue being isolated. For cross-species hybridization, low stringency conditions are preferred. For same-species hybridization, moderately stringent conditions are preferred. After successful amplification of a segment of a MO-1 homologue, that segment may be cloned, sequenced, and utilized as a probe to isolate a complete cDNA or genomic clone. This, in turn, will permit the determination of the gene's complete nucleotide sequence, the analysis of its expression, and the production of its protein product for functional analysis. In this fashion, additional nucleotide sequences encoding MO-1 or MO-1 homologues may be identified.

The above recited methods are not meant to limit the following general description of methods by which clones of genes encoding MO-1 or homologues thereof may be obtained.

Any eukaryotic cell potentially can serve as the nucleic acid source for the molecular cloning of the MO-1 gene, MO-1 cDNA or a homologue thereof. The nucleic acid sequences encoding MO-1 can be isolated from vertebrate, mammalian, human, porcine, bovine, feline, avian, equine, canine, as well as additional primate sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell, or by PCR amplification and cloning. See, for example, Sambrook et al., Molecular Cloning, A Laboratory Manual, 3d. ed., Cold Spring Harbor Laboratory Press, Cold Spring Harbor, N.Y. (2001); Glover, D. M. (ed.), DNA Cloning: A Practical Approach, 2d. ed., MRL Press, Ltd., Oxford, U.K. (1995). Clones derived from genomic DNA may contain regulatory and intron DNA regions in addition to coding regions; clones derived from cDNA will contain only exon sequences. Whatever the source, the gene may be cloned into a suitable vector for propagation of the gene.

In the cloning of the gene from genomic DNA, DNA fragments are generated, some of which will encode the desired gene. The DNA may be cleaved at specific sites using various restriction enzymes. Alternatively, one may use DNase in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the desired gene may be accomplished in a number of ways. For example, if a MO-1 gene (of any species) or its specific RNA is available and can be purified and labeled, the generated DNA fragments may be screened by nucleic acid hybridization to the labeled probe (Benton and Davis, Science 196:180 (1977); Grunstein and Hogness, Proc. Natl. Acad. Sci. U.S.A. 72:3961 (1975). Those DNA fragments with substantial homology to the probe will hybridize. It is also possible to identify the appropriate fragment by restriction enzyme digestion(s) and comparison of fragment sizes with those expected according to a known restriction map if such is available. Further selection can be carried out on the basis of the properties of the gene.

Alternatively, the presence of the gene may be detected by assays based on the physical, chemical, or immunological properties of its expressed product. For example, cDNA clones, or DNA clones that hybrid-select the proper mRNAs, can be selected that produce a protein having e.g., similar or identical electrophoretic migration, isoelectric focusing behavior, proteolytic digestion maps, substrate binding activity, or antigenic properties as known for a specific MO-1. If an antibody to a particular MO-1 is available, that MO-1 may be identified by binding of labeled antibody to the clone(s) putatively producing the MO-1 in an ELISA (enzyme-linked immunosorbent assay)-type procedure.

A MO-1 or homologue thereof can also be identified by mRNA selection by nucleic acid hybridization followed by in vitro translation. In this procedure, fragments are used to isolate complementary mRNAs by hybridization. Such DNA fragments may represent available, purified DNA of another species containing a gene encoding MO-1. Immunoprecipitation analysis or functional assays of the in vitro translation products of the isolated mRNAs identifies the mRNA and, therefore, the complementary DNA fragments that contain the desired sequences. In addition, specific mRNAs may be selected by adsorption of polysomes isolated from cells to immobilized antibodies specifically directed against a specific MO-1. A radiolabelled MO-1-encoding cDNA can be synthesized using the selected mRNA (from the adsorbed polysomes) as a template. The radiolabelled mRNA or cDNA may then be used as a probe to identify the MO-1-encoding DNA fragments from among other genomic DNA fragments.

Alternatives to isolating the MO-1 genomic DNA include, but are not limited to, chemically synthesizing the gene sequence itself from a known sequence or making cDNA to the mRNA which encodes MO-1. For example RNA for the cloning of MO-1 cDNA can be isolated from cells that express a MO-1 gene. Other methods are possible and within the scope of the invention.

The identified and isolated MO-1 or MO-1 analog-encoding gene can then be inserted into an appropriate cloning vector. A large number of vector-host systems known in the art may be used. Possible cloning vectors include, but are not limited to, plasmids or modified viruses, but the vector system must be compatible with the host cell used. Such vectors include, but are not limited to bacteriophages such as lambda derivatives, or plasmids such as pBR322, pUC plasmid derivatives, or the pBluescript vector. (Stratagene). The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini. These ligated linkers may comprise specific chemically synthesized oligonucleotides encoding restriction endonuclease recognition sequences. In an alternative method, the cleaved vector and MO-1-encoding gene or nucleic acid sequence may be modified by homopolymeric tailing. Recombinant molecules can be introduced into host cells via transformation, transfection, infection, electroporation, etc., so that many copies of the gene sequence are generated.

In an alternative method, the desired gene may be identified and isolated after insertion into a suitable cloning vector in a “shotgun” approach. Enrichment for the desired gene, for example, by size fractionization, can be done before insertion into the cloning vector.

To generate multiple copies of the isolated MO-1-encoding gene, cDNA, or synthesized DNA sequence, host cells, for example competent strains of E. Coli, may be transformed with recombinant DNA molecules incorporating said sequences according to any technique known in the art. Thus, the gene may be obtained in large quantities by growing transformants, isolating the recombinant DNA molecules from the transformants and, when necessary, retrieving the inserted gene from the isolated recombinant DNA.

5.3.2 Expression Vectors

In still another aspect, the invention provides expression vectors for expressing isolated MO-1-encoding sequences, e.g., cDNA sequences. Generally, expression vectors are recombinant polynucleotide molecules comprising expression control sequences operatively linked to a nucleotide sequence encoding a polypeptide. Expression vectors can readily be adapted for function in prokaryotes or eukaryotes by inclusion of appropriate promoters, replication sequences, selectable markers, etc. to result in stable transcription and translation of mRNA. Techniques for construction of expression vectors and expression of genes in cells comprising the expression vectors are well known in the art. See, e.g., Sambrook et al., 2001, Molecular Cloning—A Laboratory Manual, 3^{rd} edition, Cold Spring Harbor Laboratory, Cold Spring Harbor, N.Y., and Ausubel et al., eds., Current Edition, Current Protocols in Molecular Biology, Greene Publishing Associates and Wiley Interscience, NY.

Useful promoters for use in expression vectors include, but are not limited to, a metallothionein promoter, a constitutive adenovirus major late promoter, a dexamethasone-inducible MMTV promoter, a SV40 promoter, a MRP pol III promoter, a constitutive MPSV promoter, an RSV promoter, a tetracycline-inducible CMV promoter (such as the human immediate-early CMV promoter), and a constitutive CMV promoter. In one embodiment, the promoter is an adipocyte-specific promoter.

The expression vectors should contain expression and replication signals compatible with the cell in which the MO-1-encoding sequences are to be expressed. Expression vectors useful for expressing MO-1-encoding sequences include viral vectors such as retroviruses, adenoviruses and adenoassociated viruses, plasmid vectors, cosmids, and the like. Viral and plasmid vectors are preferred for transfecting the expression vectors into mammalian cells. For example, the expression vector pcDNA1 (Invitrogen, San Diego, Calif.), in which the expression control sequence comprises the CMV promoter, provides good rates of transfection and expression into such cells.

The expression vectors can be introduced into the cell for expression of the MO-1-encoding sequence by any method known to one of skill in the art without limitation. Such methods include, but are not limited to, e.g., direct uptake of the recombinant DNA molecule by a cell from solution; facilitated uptake through lipofection using, e.g., liposomes or immunoliposomes; particle-mediated transfection; etc. See, e.g., U.S. Pat. No. 5,272,065; Goeddel et al., Methods in Enzymology, vol. 185, Academic Press, Inc., CA (1990); Krieger, Gene Transfer and Expression—A Laboratory Manual, Stockton Press, New York (1990); Ausubel et al., Current Protocols In Molecular Biology, John Wiley and Sons, New York (current edition); Sambrook et al., Molecular Cloning, A Laboratory Manual, 3d. ed., Cold Spring Harbor Laboratory Press, Cold Spring Harbor, N.Y. (2001).

The expression vectors can also contain a purification moiety that simplifies isolation of the expressed protein. For example, a polyhistidine moiety of, e.g., six histidine residues, can be incorporated at the amino terminal end of the protein. The polyhistidine moiety allows convenient isolation of the protein in a single step by nickel-chelate chromatography. In certain embodiments, the purification moiety can be cleaved from the remainder of the delivery construct following purification. In other embodiments, the moiety does not interfere with the function of the functional domains of the expressed protein of the invention and thus need not be cleaved.

5.3.3 Cells

In yet another aspect, the invention provides a cell comprising a vector, e.g., an expression vector for expression of MO-1 polypeptides of the invention, or portions thereof. The cell is preferably selected for its ability to express high concentrations of the MO-1 polypeptide to facilitate subsequent purification of the polypeptide. In certain embodiments, the cell is a prokaryotic cell, for example, E. coli. In a preferred embodiment, the MO-1 polypeptide is properly folded and comprises the appropriate disulfide linkages when expressed in E. coli.

In other embodiments, the cell is a eukaryotic cell. Useful eukaryotic cells include, for example, plant, yeast and mammalian cells. Any mammalian cell known by one of skill in the art to be useful for expressing a recombinant polypeptide, without limitation, can be used to express the polypeptide of interest. For example, Chinese hamster ovary (CHO) cells can be used to express the MO-1 polypeptides of the invention. In some embodiments, the MO-1 polypeptide is expressed in adipocytes, e.g., human adipocytes. In some embodiments, the MO-1 polypeptide is labeled with a moiety to, for example, facilitate purification or identification, e.g., a FLAG tag, a GST tag, or a V-5 tag.

In yet other embodiments, the cell has been engineered to overexpress MO-1. In some embodiments, the cell expresses MO-1 at least about 10%, 20%, 30%, 40%, 50%, 60%, 70%, 80%, 90%, 100%, 150%, 200%, 300%, 400%, or 500% more than a corresponding cell that has not been engineered to overexpress MO-1. In yet other embodiments, expression of MO-1 in the cell has been silenced. In certain embodiments, expression of MO-1 is reduced by at least about 10%, 20%, 30%, 40%, 50%, 60%, 70%, 80%, 90%, 95%, 99%, or 99.9% relative to a corresponding cell where expression of MO-1 has not been silenced.

5.4 Antibodies

According to the invention, MO-1, or its fragments thereof, may be used as an immunogen to generate antibodies which immunospecifically bind MO-1 polypeptides. Such antibodies include, but are not limited to, polyclonal, monoclonal, single chain monoclonal, recombinant, chimeric, humanized, mammalian, or human antibodies.

In some embodiments, antibodies to a non-human MO-1 are produced. In certain embodiments, antibodies to mouse or rat MO-1 are produced. In other embodiments, antibodies to human MO-1 are produced. In another embodiment, antibodies are produced that specifically bind to a protein the amino acid sequence of which consists of SEQ ID NO:1. In another embodiment, antibodies to a fragment of non-human MO-1 are produced. In another embodiment, antibodies to a fragment of human MO-1 are produced. In a specific embodiment, fragments of MO-1, human or non-human, identified as containing hydrophilic regions are used as immunogens for antibody production. In a specific embodiment, a hydrophilicity analysis can be used to identify hydrophilic regions of MO-1, which are potential epitopes, and thus can be used as immunogens.

For the production of antibody, various host animals can be immunized by injection with native MO-1, or a synthetic version, or a fragment thereof. In certain embodiments, the host animal is a mammal. In some embodiments, the mammal is a rabbit, mouse, rat, goat, cow or horse.

For the production of polyclonal antibodies to MO-1, various procedures known in the art may be used. In a particular embodiment, rabbit polyclonal antibodies to an epitope of MO-1 encoded by a sequence of SEQ ID NO:2 or a subsequence thereof, can be obtained. Various adjuvants may be used to increase the immunological response, depending on the host species. Adjuvants that may be used according to the present invention include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, CpG-containing nucleic acids, and potentially useful human adjuvants such as BCG (bacille Calmette-Guerin) and Corynebacterium parvum.

For preparation of monoclonal antibodies directed toward a MO-1 polypeptide, any technique that provides for the production of antibody molecules by continuous cell lines in culture may be used. For example, monoclonal antibodies may be prepared by the hybridoma technique originally developed by Kohler and Milstein, Nature 256:495-497 (1975), as well as the trioma technique, the human B-cell hybridoma technique (Kozbor et al., Immunol. Today 4:72 (1983)), or the EBV-hybridoma technique (Cole et al., in Monoclonal Antibodies and Cancer Therapy, Alan R. Liss, Inc., pp. 77-96 (1985)).

Techniques for the production of single chain antibodies, as described in U.S. Pat. No. 4,946,778, can also be adapted to produce single chain antibodies specific to MO-1. An additional embodiment of the invention utilizes the techniques described for the construction of Fab expression libraries (Huse et al., Science 246:1275-1281 (1988)) to allow rapid and easy identification of monoclonal Fab fragments with the desired specificity for MO-1. Antibody fragments that contain the idiotype of the molecule can be generated by known techniques. For example, such fragments include but are not limited to: the F(ab′), fragment which can be produced by pepsin digestion of the antibody molecule; the Fab′ fragments which can be generated by reducing the disulfide bridges of the F(ab′), fragment, the Fab fragments which can be generated by treating the antibody molecule with papain and a reducing agent, and Fv fragments.

Techniques developed for the production of “chimeric” antibodies (Morrison et al., Proc. Natl. Acad. Sci. U.S.A. 81:6851-6855 (1984); Neuberger et al., Nature 312:604-608 (1984); Takeda et al., Nature 314:452-454 (1985)) can also be used. For example, nucleic acid sequences encoding a mouse antibody molecule specific to MO-1 are spliced to nucleic acid sequences encoding a human antibody molecule.

In addition, techniques have been developed for the production of humanized antibodies, and such humanized antibodies to MO-1 are within the scope of the present invention. See, e.g., Queen, U.S. Pat. No. 5,585,089 and Winter, U.S. Pat. No. 5,225,539. An immunoglobulin light or heavy chain variable region consists of a “framework” region interrupted by three hypervariable regions, referred to as complementarity determining regions (CDRs). The extent of the framework region and CDRs have been precisely defined. See, Sequences of Proteins of Immunological Interest, Kabat, E. et al., U.S. Department of Health and Human Services (1983). Briefly, humanized antibodies are antibody molecules from non-human species having one or more CDRs from the non-human species and a framework region from a human immunoglobulin molecule.

Human antibodies may be used and can be obtained by using human hybridomas (Cote et al., Proc. Natl. Acad. Sci. U.S.A., 80:2026-2030 (1983)) or by transforming human B cells with EBV virus in vitro (Cole et al., in Monoclonal Antibodies and Cancer Therapy, Alan R. Liss, pp. 77-96 (1985)).

In the production of antibodies, screening for the desired antibody can be accomplished by techniques known in the art, e.g. ELISA (enzyme-linked immunosorbent assay), RIA (radioimmunoassay) or RIBA (recombinant immunoblot assay). For example, to select antibodies which recognize a specific domain of MO-1, one may assay generated hybridomas for a product which binds to a MO-1 fragment containing such domain. For selection of an antibody that specifically binds a first MO-1 homologue but which does not specifically bind a second, different MO-1 homologue, one can select on the basis of positive binding to the first MO-1 homologue and a lack of binding to the second MO-1 homologue.

Antibodies specific to a domain of MO-1 or a homologue thereof are also provided. The foregoing antibodies can be used in methods known in the art relating to the localization and activity of the MO-1 of the invention, e.g., for imaging these proteins, measuring levels thereof in appropriate physiological samples, in diagnostic methods, etc.

5.5 Transgenic MO-1 Animals

Transgenic animals are useful, e.g., for identifying and/or evaluating modulators of MO-1 activity, and, as such, for identifying modulators to be tested for that ability. Transgenes direct the expression of an encoded gene product in one or more cell types or tissues of the transgenic animal. In some embodiments, transgenes prevent the expression of a naturally encoded gene product in one or more cell types or tissues (a “knockout” transgenic animal). In some embodiments, transgenes serve as a marker or indicator of an integration, chromosomal location, or region of recombination (e.g., cre/loxP mice).

A transgenic animal can be created by introducing a nucleic acid of the invention into the male pronuclei of a fertilized oocyte (e.g., by microinjection, retroviral infection) and allowing the oocyte to develop in a pseudopregnant female foster animal (PFFA). The MO-1 sequences can be introduced as a transgene into the genome of a non-human animal. In some embodiments, the MO-1 sequence is the human MO-1 sequence (SEQ ID NO:2). In other embodiments, a homologue of MO-1 can be used as a transgene. Intronic sequences and polyadenylation signals can also be included in the transgene to increase transgene expression. Tissue-specific regulatory sequences can be operably-linked to the MO-1 transgene to direct expression of MO-1 to particular cells. Methods for generating transgenic animals via embryo manipulation and microinjection, particularly animals such as mice, have become conventional in the art, e.g., Evans et al., U.S. Pat. No. 4,870,009 (1994); Leder and Stewart, U.S. Pat. No. 4,736,866, 1988; Wagner and Hoppe, U.S. Pat. No. 4,873,191 (1989). Other non-mice transgenic animals may be made by similar methods. A transgenic founder animal, which can be used to breed additional transgenic animals, can be identified based upon the presence of the transgene in its genome and/or expression of the transgene mRNA in tissues or cells of the animal. Transgenic MO-1 animals can be bred to other transgenic animals carrying other transgenes.

To create a homologous recombinant animal, a vector containing at least a portion of MO-1 into which a deletion, addition or substitution may be introduced to thereby alter, e.g., functionally disrupt MO-1 expression. In some embodiments, the vector may contain a neomycin cassette inserted in reverse orientation relative to MO-1 transcription to functionally disrupt MO-1. MO-1 can be a human gene (e.g., SEQ ID NO:2 or 3), or other MO-1 homologue. In one approach, a knockout vector functionally disrupts the endogenous MO-1 gene upon homologous recombination, and thus a non-functional MO-1 protein, if any, is expressed.

Alternatively, the vector can be designed such that, upon homologous recombination, the endogenous MO-1 is mutated or otherwise altered but still encodes functional protein (e.g., the upstream regulatory region can be altered to thereby alter the expression of endogenous MO-1). In this type of homologous recombination vector, the altered portion of the MO-1 sequence is flanked at its 5′- and 3′-termini by additional nucleic acid sequence of MO-1 to allow for homologous recombination to occur between the exogenous MO-1 sequence carried by the vector and an endogenous MO-1 sequence in an embryonic stem cell. The additional flanking MO-1 sequence is sufficient to engender homologous recombination with endogenous MO-1. Typically, several kilobases of flanking DNA (both at the 5′- and 3′-termini) are included in the vector (see Thomas and Capecchi, Cell 51:503-512 (1987)).

The vector can then be introduced into an embryonic stem cell line (e.g., by electroporation), and cells in which the introduced MO-1 sequence has homologously-recombined with the endogenous MO-1 sequence are selected (Li et al., Cell 69:915-926 (1992)).

Selected cells are then injected into a blastocyst of an animal (e.g., a mouse) to form aggregation chimeras (see Bradley, Teratocarcinomas and Embryonic Stem Cells: A Practical Approach, Oxford University Press, Inc., Oxford (1987)). A chimeric embryo can then be implanted into a suitable PFFA, wherein the embryo is brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described (Berns et al., WO 93/04169, 1993; Kucherlapati et al., WO 91/01140, 1991; Le Mouellic and Brullet, WO 90/11354, 1990).

Alternatively, transgenic animals that contain selected systems that allow for regulated expression of the transgene can be produced. An example of such a system is the cre/loxP recombinase system of bacteriophage P1 (Lakso et al., Proc. Natl. Acad. Sci. USA 89:6232-6236 (1992)). Another recombinase system is the FLP recombinase system of Saccharomyces cerevisiae (O'Gorman et al., Science 251:1351-1355 (1991)). If a cre/loxP recombinase system is used to regulate expression of the transgene, animals containing transgenes encoding both the Cre recombinase and a selected protein are required. Such animals can be produced as “double” transgenic animals, by mating an animal containing a transgene encoding a selected protein to another containing a transgene encoding a recombinase.

Clones of transgenic animals can also be produced (Wilmut et al., Nature 385:810-813 (1997)). In brief, a cell from a transgenic animal can be isolated and induced to exit the growth cycle and enter G₀ phase. The quiescent cell can then be fused to an enucleated oocyte from an animal of the same species from which the quiescent cell is isolated. The reconstructed oocyte is then cultured to develop to a morula or blastocyte and then transferred to a PFFA. The offspring borne of this female foster animal will be a clone of the “parent” transgenic animal.

In certain embodiments, the transgeneic animal exhibits a phenotype associated with altered MO-1 activity, e.g., obesity.

5.6 Methods of Screening for Modulators of MO-1 Activity

The present invention also provides methods of identifying a compound that modulates the activity of MO-1 in a cell or tissue of interest. A compound may modulate MO-1 activity by affecting, for example: (1) the number of copies of the MO-1 gene in the cell (amplifiers and deamplifiers); (2) increasing or decreasing transcription of the MO-1 gene (transcription up-regulators and down-regulators); (3) by increasing or decreasing the translation of the MO-1 mRNA into protein (translation up regulators and down regulators); (4) by increasing or decreasing the activity of the MO-1 protein (agonists and antagonists), or 5) by facilitating the proper folding of the MO-1 protein (pharmacological chaperonins). To identify compounds that affect MO-1 at the DNA, RNA, and protein levels, cells or organisms are contacted with a candidate compound and the corresponding change in MO-1 DNA, RNA or protein may be assessed. For DNA amplifiers or deamplifiers, the amount of MO-1 DNA may be measured. For those compounds that are transcription up-regulators and down-regulators, the amount of MO-1 mRNA may be measured. Alternatively, the MO-1 promoter sequence may be operably linked to a reporter gene, and potential transcriptional modulators of MO-1 may be assayed by measuring reporter gene activity in the presence and absence of the compound. For translational up- and down-regulators, the amount of MO-1 polypeptide may be measured. Alternatively, changes in MO-1 biological activity, as measured by the techniques described below, may be an indirect indicator of the ability of a compound to modulate MO-1 translation.

In one embodiment, the cell or tissue useful for the methods described herein expresses a MO-1 polypeptide from an endogenous copy of the MO-1 gene. In another embodiment, the cell or tissue expresses a MO-1 polypeptide following transient or stable transformation with a nucleic acid encoding a MO-1 polypeptide of the present invention. Any mammalian cell known by one of skill in the art to be useful for expressing a recombinant polypeptide, without limitation, can be used to express a MO-1 polypeptide useful for the methods described herein.

In one embodiment, the method of identifying a compound that modulates the activity of MO-1 comprises determining a first level of MO-1 activity in a cell or tissue that expresses a MO-1 polypeptide, contacting said cell or tissue with a test compound, then determining a second level of MO-1 activity in said cell or tissue. A difference in the first level and second level of MO-1 activity is indicative of the ability of the test compound to modulate MO-1 activity. In one embodiment, a compound may have agonistic activity if the second level of MO-1 activity is greater than the first level of MO-1 activity. In certain embodiments, agonistic activity comprises at least about a 2, 4, 6, 8, 10, or greater fold increase in the second level of MO-1 activity compared to the first level of MO-1 activity. In another embodiment, a compound may have antagonistic activity if the second level of MO-1 activity is less than the first level of MO-1 activity. In certain embodiments, antagonistic activity comprises at least about a 2, 4, 6, 8, 10, or greater fold decrease in the second level of MO-1 activity compared to the first level of MO-1 activity.

In another embodiment, the invention provides a method of identifying a compound that modulates the activity of MO-1 in a cell or tissue expressing a MO-1 polypeptide, comprising contacting said cell or tissue with a test compound and determining a level of MO-1 in said cell or tissue. The difference in this level and a standard or baseline level of MO-1 activity in a comparable cell or tissue, e.g., a control cell or tissue not contacted with the test compound, is indicative of the ability of said test compound to modulate MO-1 activity. In one embodiment, a compound may have agonistic activity if the level of MO-1 activity in the cell or tissue contacted with said compound is greater than the level of MO-1 activity in the control cell or tissue. In certain embodiments, agonistic activity comprises at least about a 2-, 4-, 6-, 8-, 10-, or greater fold increase in the level of MO-1 activity of a cell or tissue contacted with the test compound compared to the level of MO-1 activity in the control cell or tissue. In another embodiment, a compound may have antagonistic activity if the level of MO-1 activity in the cell or tissue contacted with said compound is less than the level of MO-1 activity in the control cell or tissue. In certain embodiments, antagonistic activity comprises at least about a 2-, 4-, 6-, 8-, 10-, or greater fold decrease in the level of MO-1 activity of a cell or tissue contacted with the test compound compared to the level of MO-1 activity in the control cell or tissue.

The present invention also provides methods of identifying a compound that modulates the activity of MO-1 in a transgenic non-human animal which expresses a MO-1 polypeptide, comprising administering the compound to said animal and assessing the animal for an alteration in metabolic function affected by the compound. Metabolic function may be assessed through the measurement of glucose concentrations, lipid concentrations, mass of the animal, and the like.

The present invention also provides methods of identifying compounds that specifically bind to MO-1 nucleic acids or polypeptides and thus have potential use as agonists or antagonists of MO-1. In certain embodiments, such compounds may affect glucose concentrations, lipid concentrations, mass of the animal, etc. In a preferred embodiment, assays are performed to screen for compounds having potential utility as therapies for metabolic disorders or lead compounds for drug development. The invention thus provides assays to detect compounds that specifically bind to MO-1 nucleic acids or polypeptides. For example, recombinant cells expressing MO-1 nucleic acids can be used to recombinantly produce MO-1 polypeptides for use in these assays, e.g., to screen for compounds that bind to MO-1 polypeptides. Said compounds (e.g., putative binding partners of MO-1) are contacted with a MO-1 polypeptide or a fragment thereof under conditions conducive to binding, and compounds that specifically bind to MO-1 are identified. Similar methods can be used to screen for compounds that bind to MO-1 nucleic acids. Methods that can be used to carry out the foregoing are commonly known in the art.

In various embodiments, the MO-1-modulating compound is a protein, for example, an antibody; a nucleic acid; or a small molecule. As used herein, the term “small molecule” includes, but is not limited to, organic or inorganic compounds (i.e., including heteroorganic and organometallic compounds) having a molecular weight less than 10,000 grams per mole, organic or inorganic compounds having a molecular weight less than 5,000 grams per mole, organic or inorganic compounds having a molecular weight less than 1,000 grams per mole, organic or inorganic compounds having a molecular weight less than 500 grams per mole, organic or inorganic compounds having a molecular weight less than 100 grams per mole, and salts, esters, and other pharmaceutically acceptable forms of such compounds. Salts, esters, and other pharmaceutically acceptable forms of such compounds are also encompassed.

By way of example, diversity libraries, such as random or combinatorial peptide or nonpeptide libraries can be screened for molecules that specifically bind to MO-1. Many libraries are known in the art that can be used, e.g., chemically synthesized libraries, recombinant (e.g., phage display libraries), and in vitro translation-based libraries.

Examples of chemically synthesized libraries are described in Fodor et al., Science 251:767-773 (1991); Houghten et al., Nature 354:84-86 (1991); Lam et al., Nature 354:82-84 (1991); Medynski, Bio/Technology 12:709-710 (1994); Gallop et al., J. Medicinal Chemistry 37 (9):1233-1251 (1994); Ohlmeyer et al., Proc. Natl. Acad. Sci. U.S.A. 90:10922-10926 (1993); Erb et al., Proc. Natl. Acad. Sci. U.S.A. 91:11422-11426 (1994); Houghten et al., Biotechniques 13:412 (1992); Jayawickreme et al., Proc. Natl. Acad. Sci. U.S.A. 91:1614-1618 (1994); Salmon et al., Proc. Natl. Acad. Sci. U.S.A. 90:11708-11712 (1993); PCT Publication No. WO 93/20242; and Brenner and Lerner, Proc. Natl. Acad. Sci. U.S.A. 89:5381-5383 (1992).

Examples of phage display libraries are described in Scott and Smith, Science 249:386-390 (1990); Devlin et al., Science, 249:404-406 (1990); Christian, R. B., et al., J. Mol. Biol. 227:711-718 (1992)); Lenstra, J. Immunol. Meth. 152:149-157 (1992); Kay et al., Gene 128:59-65 (1993); and PCT Publication No. WO 94/18318, published Aug. 18, 1994. In vitro translation-based libraries include but are not limited to those described in PCT Publication No. WO 91/05058, published Apr. 18, 1991; and Mattheakis et al., Proc. Natl. Acad. Sci. U.S.A. 91:9022-9026 (1994).

By way of examples of non-peptide libraries, a benzodiazepine library (see e.g., Bunin et al., Proc. Natl. Acad. Sci. U.S.A. 91:4708-4712 (1994)) can be adapted for use. Peptoid libraries (Simon et al., Proc. Natl. Acad. Sci. U.S.A. 89:9367-9371 (1992)) can also be used. Another example of a library that can be used, in which the amide functionalities in peptides have been permethylated to generate a chemically transformed combinatorial library, is described by Ostresh et al., Proc. Natl. Acad. Sci. U.S.A. 91:11138-11142 (1994).

Screening the libraries can be accomplished by any of a variety of commonly known methods. See, e.g., the following references, which disclose screening of peptide libraries: Parmley and Smith, Adv. Exp. Med. Biol. 251:215-218 (1989); Scott and Smith, Science 249:386-390 (1990); Fowlkes et al., Bio/Techniques 13:422-427 (1992); Oldenburg et al., Proc. Natl. Acad. Sci. U.S.A. 89:5393-5397 (1992); Yu et al., Cell 76:933-945 (1994); Staudt et al., Science 241:577-580 (1988); Bock et al., Nature 355:564-566 (1992); Tuerk et al., Proc. Natl. Acad. Sci. U.S.A. 89:6988-6992 (1992); Ellington et al., Nature 355:850-852 (1992); U.S. Pat. No. 5,096,815, U.S. Pat. No. 5,223,409, and U.S. Pat. No. 5,198,346; Rebar and Pabo, Science 263:671-673 (1993); and PCT Publication No. WO 94/18318, published Aug. 8, 1994.

In a specific embodiment, screening can be carried out by contacting the library members with MO-1 polypeptide (or nucleic acid) immobilized on a solid phase and harvesting those library members that bind to the protein (or nucleic acid). Examples of such screening methods, termed “panning” techniques are described by way of example in Parmley and Smith, Gene 73:305-318 (1988); Fowlkes et al., Bio/Techniques 13:422-427 (1992); PCT Publication No. WO 94/18318; and in references cited herein above.

In another embodiment, the two-hybrid system for selecting interacting proteins in yeast (Fields and Song, Nature 340:245-246 (1989); Chien et al., Proc. Natl. Acad. Sci. U.S.A. 88:9578-9582 (1991)) can be used to identify molecules that specifically bind to MO-1 protein or an analog thereof.

In another embodiment, screening can be carried out by creating a peptide library in a prokaryotic or eukaryotic cell, such that the library proteins are expressed on the cells' surface, followed by contacting the cell surface with MO-1 and determining whether binding has taken place. Alternatively, the cells are transformed with a nucleic acid encoding MO-1, such that MO-1 is expressed on the cells' surface. The cells are then contacted with a potential agonist or antagonist, and binding, or lack thereof, is determined. In a specific embodiment of the foregoing, the potential agonist or antagonist is expressed in the same or a different cell such that the potential agonist or antagonist is expressed on the cells' surface.

In another embodiment, screening can be carried out by assessing modulation (e.g., an increase or decrease) of binding of MO-1 to another polypeptide. In certain embodiments, the polypeptide is SCP2, CYP2B6, MTO1-like or IRAP.

As would clearly be understood by a person of ordinary skill in the art, any and/or all of the embodiments disclosed herein for identifying an agent, drug, or compound that can modulate the activity of MO-1, including such procedures that incorporate rational drug design, as disclosed herein, can be combined to form additional drug screens and assays, all of which are contemplated by the present invention.

5.7 Diagnostic Methods

The present invention also pertains to the field of predictive medicine in which diagnostic and prognostic assays are used for prognostic (predictive) purposes to treat an individual prophylactically. Accordingly, one aspect of the invention relates to diagnostic assays for determining MO-1 nucleic acid expression as well as MO-1 activity in the context of a biological sample (e.g., blood, serum, cells, tissue) to determine whether an individual is afflicted with a disease or disorder, or is at risk of developing a disorder. Such a disease or disorder may be associated with aberrant MO-1 expression or activity, and can include, but is not limited to, obesity or other related metabolic disorders. The invention also provides for prognostic assays for determining whether an individual is at risk of developing a disorder associated with MO-1 nucleic acid expression or activity. For example, mutations in MO-1 can be assayed in a biological sample. Such assays can be used for prognostic or predictive purpose to prophylactically treat an individual prior to the onset of a disorder characterized by or associated with aberrant MO-1 nucleic acid expression or biological activity.

5.7.1 Diagnostic Assays

An exemplary method for detecting the presence or absence of MO-1 in a biological sample involves obtaining a biological sample from a subject and contacting the biological sample with a compound or an agent capable of detecting MO-1 nucleic acid (e.g., mRNA, genomic DNA) such that the presence of MO-1 is confirmed in the sample. An agent for detecting MO-1 mRNA or genomic DNA is a labeled nucleic acid probe that can hybridize to MO-1 mRNA or genomic DNA. The nucleic acid probe can be, for example, a full-length MO-1 nucleic acid, such as the nucleic acid of SEQ ID NOS:2 or 3, or a portion thereof. In some embodiments, the nucleic acid probe is an oligonucleotide of at least 15, 30, 50, 100, 250 or 500 nucleotides in length and is sufficient to specifically hybridize under stringent conditions to MO-1 mRNA or genomic DNA. In certain embodiments, a mutation resulting in a premature stop codon at amino acid position 82 of the MO-1 protein is detected.

An agent for detecting MO-1 polypeptide can be an antibody capable of binding to MO-1, preferably an antibody with a detectable label. Antibodies can be polyclonal or monoclonal. An intact antibody or an antibody fragment, e.g., a Fab fragment, can be used. A labeled probe or antibody may be coupled (i.e., physically linked) to a detectable substance, or an indirect detection method may be employed wherein the probe or antibody is detected via reactivity with a directly labeled secondary reagent. Examples of indirect labeling include detection of a primary antibody using a fluorescently labeled secondary antibody, or end-labeling of a DNA probe with biotin such that it can be detected with fluorescently-labeled streptavidin.

The detection method of the invention can be used to detect MO-1 mRNA, protein, or genomic DNA in a biological sample in vitro as well as in vivo. For example, in vitro techniques for detection of MO-1 mRNA include Northern hybridizations and in situ hybridizations. In vitro techniques for detection of MO-1 polypeptide include enzyme linked immunosorbent assays (ELISAs), Western blots, immunoprecipitations, and immunofluorescence. In vitro techniques for detection of MO-1 genomic DNA include Southern hybridizations and fluorescence in situ hybridization (FISH). Furthermore, in vivo techniques for detecting MO-1 include introducing into a subject a labeled anti-MO-1 antibody. For example, the antibody can be labeled with a radioactive marker whose presence and location in a subject can be detected by standard imaging techniques.

In one embodiment, the biological sample from the subject contains protein molecules, and/or mRNA molecules, and/or genomic DNA molecules. In certain embodiments, the biological sample is blood.

In another embodiment, the methods further involve obtaining a biological sample from a subject to provide a control, contacting the sample with a compound or agent to detect MO-1 mRNA or genomic DNA, and comparing the presence of MO-1 mRNA or genomic DNA in the control sample with the presence of MO-1 mRNA or genomic DNA in the test sample.

In another embodiment, the methods comprise assessing a biological activity of MO-1. For example, lipid concentration, glucose concentration, cellular proliferation, and expression levels of genes whose expression is modulated by MO-1 can be assessed. Accordingly, in certain embodiments, decreased lipid concentrations reflects decreased MO-1 activity. In certain embodiments, increased lipid concentrations reflects decreased MO-1 activity. In certain embodiments, increased lipid concentrations reflects increased MO-1 activity. In certain embodiments, decreased lipid concentrations reflects increased MO-1 activity. In certain embodiments, decreased glucose concentrations reflects decreased MO-1 activity. In certain embodiments, increased glucose concentrations reflects decreased MO-1 activity. In certain embodiments, increased glucose concentrations reflects increased MO-1 activity. In certain embodiments, decreased glucose concentrations reflects increased MO-1 activity.

In certain embodiments, decreased cellular proliferation reflects decreased MO-1 activity. In certain embodiments, increased cellular proliferation reflects decreased MO-1 activity. In certain embodiments, increased cellular proliferation reflects increased MO-1 activity. In certain embodiments, decreased cellular proliferation reflects increased MO-1 activity. In certain embodiments, the affected cells are adipocytes.

5.7.2 Prognostic Assays

The diagnostic methods described herein can be further utilized to identify subjects having, or who are at risk of developing, a disease or disorder associated with aberrant MO-1 expression or activity. Such a disease or disorder may include, but is not limited to, metabolic disorders such as, e.g., obesity. The invention provides a method for identifying a disease or disorder associated with aberrant MO-1 expression or activity in which a test sample is obtained from a subject and MO-1 nucleic acid (e.g., mRNA, genomic DNA) is detected. A test sample is a biological sample obtained from a subject. For example, a test sample can be a biological fluid (e.g., serum), cell sample, or tissue.

Prognostic assays can be used to determine whether a subject can be administered a modality (e.g., an agonist, antagonist, peptidomimetic, protein, peptide, nucleic acid, small molecule, food, etc.) to treat a disease or disorder associated with aberrant MO-1 expression or activity. Such methods can be used to determine whether a subject can be effectively treated with an agent for a disorder. The invention provides methods for determining whether a subject can be effectively treated with an agent for a disorder associated with aberrant MO-1 expression or activity in which a test sample is obtained and MO-1 nucleic acid is detected (e.g., where the presence of MO-1 nucleic acid is diagnostic for a subject that can be administered the agent to treat a disorder associated with aberrant MO-1 expression or activity).

The methods of the invention can also be used to detect genetic lesions in a MO-1 gene to determine if a subject with the genetic lesion is at risk for a disorder, including but not limited to obesity. Methods include detecting, in a sample from the subject, the presence or absence of a genetic lesion characterized by an alteration affecting the integrity of a gene encoding a MO-1 polypeptide, or the mis-expression of a MO-1 gene. Such genetic lesions can be detected by ascertaining: (1) a deletion of one or more nucleotides from the MO-1 gene; (2) an addition of one or more nucleotides to the MO-1 gene; (3) a substitution of one or more nucleotides in the MO-1 gene; (4) a chromosomal rearrangement of a MO-1 gene; (5) an alteration in the level of MO-1 mRNA transcripts; (6) aberrant modification of a MO-1 gene, such as a change in genomic DNA methylation; (7) the presence of a non-wild-type splicing pattern of a MO-1 mRNA transcript, (8) a non-wild-type level of a MO-1 polypeptide; (9) allelic loss of MO-1; (10) inappropriate post-translational modification of a MO-1 polypeptide; and/or (11) a single-nucleotide polymorphism associated with a particular MO-1-related phenotype. In one embodiment, the genetic lesion is a mutation causing premature truncation of a MO-1 protein. There are a large number of known assay techniques that can be used to detect lesions in MO-1. Any biological sample containing nucleated cells may be used. In some embodiments, the biological sample can be a pre-natal sample, obtained, for example, by amniocentesis or chlorionic vili sampling (CVS).

Detection of genetic lesions of MO-1 may employ any technique known in the art. In certain embodiments, lesion detection may employ a nucleic acid probe/primer in a polymerase chain reaction (PCR) reaction such as anchor PCR or rapid amplification of cDNA ends (RACE) PCR. This method may include collecting a sample from a patient, isolating nucleic acids from the sample, contacting the nucleic acids with one or more nucleic acid primers that specifically hybridize to MO-1 nucleic acid under conditions such that hybridization and amplification of the MO-1 sequence (if present) occurs, and detecting the presence or absence of an amplification product, or detecting the size of the amplification product and comparing the length to a control sample. It is anticipated that PCR may be desirable to use as a preliminary amplification step in conjunction with any of the techniques used for detecting mutations described herein.

Mutations in a MO-1 gene from a sample can also be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicate mutations in the sample DNA. Moreover, the use of sequence specific ribozymes can be used to score for the presence of specific mutations by development or loss of a ribozyme cleavage site.

Furthermore, hybridizing a sample and control nucleic acids, e.g., DNA or RNA, to high-density arrays containing hundreds or thousands of oligonucleotides probes can identify genetic mutations in MO-1 (see Cronin et al., Hum. Mutat. 7:244-255 (1996); Kozal et al., Nat. Med. 2:753-759 (1996)). For example, genetic mutations in MO-1 can be identified in two-dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the MO-1 gene and detect mutations by comparing the sequence of the sample MO-1 sequence with the corresponding wild-type (control) sequence. Examples of sequencing reactions include those based on classic techniques (see Maxam and Gilbert, Proc. Natl. Acad. Sci USA 74:560-564 (1977); Sanger et al., Natl. Acad. Sci USA 74:5463-5367 (1977)). Any of a variety of automated sequencing procedures can be used for performing diagnostic assays of the present invention (see Naeve et al., Biotechniques 19:448-453 (1995)) including sequencing by mass spectrometry (Cohen et al., Adv. Chromatogr. 36:127-162 (1996); Griffin and Griffin, Appl. Biochem. Biotechnol. 38:147-159 (1993)).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (see Saiki et al., Nature 324:163-166 (1986); Saiki et al., Proc. Natl. Acad. Sci. USA 86:6230-6234 (1989)). Such allele-specific oligonucleotides are hybridized to PCR-amplified target DNA or a number of different mutations when the oligonucleotides are attached to the hybridizing membrane and hybridized with labeled target DNA.

In yet other embodiments, a single-nucleotide polymorphism (SMP) associated with altered MO-1 expression and/or activity can be detected. In certain embodiments, the SNP is −171C>G, −170G>A, −79insA, IVS2 +66delCT, g.168 G>A syn, or g.471A>G, syn. In certain embodiments, the SNP is −171C>G. In certain embodiments, the SNP is −170G>A. In certain embodiments, the SNP is −79insA. In certain embodiments, the SNP is IVS2 +66delCT. In certain embodiments, the SNP is g.168 G>A syn. In certain embodiments, the SNP is g.471A>G, syn.

5.8 Compositions

The invention provides methods of treatment (and prophylaxis) by administration to a subject of an effective amount of a therapeutic of the invention, e.g., a MO-1 polypeptide, a derivative thereof, a MO-1 nucleic acid that expresses a MO-1 polypeptide, etc. In a preferred aspect, the therapeutic is substantially purified. The subject is preferably an animal, including but not limited to animals such as cows, pigs, horses, chickens, cats, dogs, etc., and is preferably a mammal, and most preferably human. In a specific embodiment, a non-human mammal is the subject. Formulations and methods of administration that can be employed can be selected from among those described herein below.

Various delivery systems are known and can be used to administer a therapeutic of the invention, e.g., encapsulation in liposomes, microparticles, microcapsules, recombinant cells capable of expressing the therapeutic, receptor-mediated endocytosis (see, e.g., Wu and Wu, J. Biol. Chem. 262:4429-4432 (1987)), construction of a therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the therapeutic can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 317-372, 353-365 (1989)).

In yet another embodiment, the therapeutic can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability: Drug Product Design and Performance, Smolen and Ball (eds.), Wiley, New York (1984); Ranger and Pewas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

In a specific embodiment where the therapeutic is a nucleic acid encoding a protein therapeutic (e.g., SEQ ID NO:1), the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, DuPont), or coating with lipids or cell-surface receptors or transfecting agents, or by administering it in linkage to a homeobox-like peptide which is known to enter the nucleus (see e.g., Joliot et al., Proc. Natl. Acad. Sci. U.S.A. 88:1864-1868 (1991)), etc. Alternatively, a nucleic acid therapeutic can be introduced intracellularly and incorporated within host cell DNA for expression, by homologous recombination. In another embodiment, the nucleic acid therapeutic can act by altering, e.g., increasing or decreasing, the expression of endogenous MO-1 from the genome of the subject to be treated.

The present invention also provides pharmaceutical compositions. Such compositions comprise a therapeutically effective amount of a therapeutic, and a pharmaceutically acceptable carrier. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The therapeutics of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

5.9 Kits

The pharmaceutical compositions can be included in a kit, container, pack, or dispenser together with instructions for administration. When the invention is supplied as a kit, the different components of the composition may be packaged in separate containers and admixed immediately before use. Such packaging of the components separately may permit long-term storage without losing the active components' functions.

Kits may also include reagents in separate containers that facilitate the execution of a specific test, such as diagnostic tests or tissue typing. For example, MO-1 DNA templates and suitable primers may be supplied for internal controls.

5.9.1 Containers or Vessels

The reagents included in the kits can be supplied in containers of any sort such that the life of the different components are preserved, and are not adsorbed or altered by the materials of the container. For example, sealed glass ampules may contain lyophilized luciferase or buffer that have been packaged under a neutral, non-reacting gas, such as nitrogen. Ampoules may consist of any suitable material, such as glass, organic polymers, such as polycarbonate, polystyrene, etc., ceramic, metal or any other material typically employed to hold reagents. Other examples of suitable containers include simple bottles that may be fabricated from similar substances as ampules, and envelopes, that may consist of foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

5.9.2 Instructional Materials

Kits may also be supplied with instructional materials. Instructions may be printed on paper or other substrate, and/or may be supplied as an electronic-readable medium, such as a floppy disc, CD-ROM, DVD-ROM, Zip disc, videotape, audio tape, etc. Detailed instructions may not be physically associated with the kit; instead, a user may be directed to an internet web site specified by the manufacturer or distributor of the kit, or supplied as electronic mail.

5.10 Methods of Treatment

The invention provides for both prophylactic and therapeutic methods of treating a subject at risk for (or susceptible to) a disorder or having a disorder associated with aberrant MO-1 expression or activity. Exemplary disorders are characterized by abnormal metabolic function, including, but not limited to, diabetes, type II diabetes, obesity, morbid obesity, hyperglycemia, insulin resistance, hyperinsulinemia, hypercholesterolemia, hypertension, hyperlipoproteinemia, hyperlipidemia, hypertriglylceridemia and dyslipidemia, and the like.

5.10.1 Diseases and Disorders

Diseases and disorders that are characterized by increased MO-1 levels or biological activity may be treated with therapeutics that antagonize (i.e., reduce or inhibit) activity. Antagonists may be administered in a therapeutic or prophylactic manner. Therapeutics that may be used include: (1) MO-1 peptides, or analogs, derivatives, fragments or homologues thereof; (2) Abs to a MO-1 peptide; (3) MO-1 nucleic acids; (4) administration of antisense nucleic acid, including, for example, siRNAs, and nucleic acids that are “dysfunctional” (i.e., due to a heterologous insertion within the coding sequences) that are used to eliminate endogenous function of MO-1 by homologous recombination (Capecchi, Science 244:1288-1292 (1989)); or (5) modulators (i.e., inhibitors, agonists and antagonists, including additional peptide mimetic of the invention or Abs specific to MO-1) that alter the interaction between MO-1 and its binding partner(s), e.g., IRAP and SCP2.

Diseases and disorders that are characterized by decreased MO-1 levels or biological activity may be treated with therapeutics that increase (i.e., are agonists to) activity. Therapeutics that upregulate activity may be administered therapeutically or prophylactically. Therapeutics that may be used include peptides, or analogs, derivatives, fragments or homologues thereof; or an agonist that increases bioavailability. Alternately, a nucleic acid therapeutic that increases expression of MO-1 provided either exogenously or endogenously in the subject's genome can be used.

Increased or decreased levels can be readily detected by quantifying peptide and/or RNA, by obtaining a patient tissue sample (e.g., from bleed or biopsy tissue) and assaying in vitro for RNA or peptide levels, structure and/or activity of the expressed peptides (or MO-1 mRNAs). Methods include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

5.10.2 Prophylactic Methods

The invention provides a method for preventing, in a subject, a disease or condition associated with an aberrant MO-1 expression or activity, by administering an agent that modulates MO-1 expression or at least one MO-1 activity. Subjects at risk for a disease that is caused or contributed to by aberrant MO-1 expression or activity can be identified by, for example, any or a combination of diagnostic or prognostic assays. Administration of a prophylactic agent can occur prior to the manifestation of symptoms characteristic of the MO-1 aberrancy, such that a disease or disorder is prevented or, alternatively, delayed in its progression. In a specific embodiment of the invention, ventricular muscle cell hypertrophy is prevented or delayed by administration of said prophylactic agent. Depending on the type of MO-1 aberrancy, for example, a MO-1 agonist or MO-1 antagonist can be used to treat the subject. The appropriate agent can be determined based on screening assays.

5.10.3 Therapeutic Methods

Another aspect of the invention pertains to methods of modulating MO-1 expression or activity for therapeutic purposes. The modulatory method of the invention involves contacting a cell with an agent that modulates one or more of the activities of MO-1 activity associated with the cell. An agent that modulates MO-1 activity can be a nucleic acid or a protein, a naturally occurring cognate ligand of MO-1, a peptide, a MO-1 peptidomimetic, an aptamer, or other small molecule. The agent may stimulate MO-1 activity. Examples of such stimulatory agents include active MO-1 and a MO-1 nucleic acid molecule that has been introduced into the cell. Stimulation of MO-1 activity is desirable in situations in which MO-1 is abnormally down-regulated and/or in which increased MO-1 activity is likely to have a beneficial effect.

In other embodiments, the MO-1-modulating agent inhibits MO-1 activity. Examples of inhibitory agents include anti-MO-1 Abs, or an inhibitory nucleic acid molecule. For example, the nucleic acid molecule may comprise an antisense oligonucleotide, an aptamer, or an inhibitory/interfering RNA (e.g., a small inhibitory/interfering RNA. Methods for screening for, identifying and making these nucleic acid modulators are known in the art.

In some embodiments, RNA interference (RNAi) (see, e.g. Chuang et al., Proc. Natl. Acad. Sci. U.S.A. 97:4985 (2000)) can be employed to inhibit the expression of a gene encoding MO-1. Interfering RNA (RNAi) fragments, particularly double-stranded (ds) RNAi, can be used to generate loss-of-MO-1 function. Methods relating to the use of RNAi to silence genes in organisms, including mammals, C. elegans, Drosophila, plants, and humans are known (see, e.g., Fire et al., Nature 391:806-811 (1998); Fire, Trends Genet. 15:358-363 (1999); Sharp, Genes Dev. 15:485-490 (2001); Hammond, et al., Nature Rev. Genet. 2:1110-1119 (2001); Tuschl, Chem. Biochem. 2:239-245 (2001); Hamilton et al., Science 286:950-952 (1999); Hammond et al., Nature 404:293-296 (2000); Zamore et al., Cell 101:25-33 (2000); Bernstein et al., Nature 409: 363-366 (2001); Elbashir et al., Genes Dev. 15:188 200 (2001); Elbashir et al. Nature 411:494-498 (2001); International PCT application No. WO 01/29058; and International PCT application No. WO 99/32619), the contents of which are incorporated by reference. Double-stranded RNA (dsRNA)-expressing constructs are introduced into a host using a replicable vector that remains episomal or integrates into the genome. By selecting appropriate sequences, expression of dsRNA can interfere with accumulation of endogenous mRNA encoding MO-1.

Modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a MO-1 or nucleic acid molecule. In one embodiment, the method involves administering an agent (e.g., an agent identified by a screening assay), or combination of agents that modulates (e.g., up-regulates or down-regulates) MO-1 expression or activity. In another embodiment, the method involves administering a MO-1 or nucleic acid molecule as therapy to compensate for reduced or aberrant MO-1 expression or activity.

5.10.4 Determination of the Biological Effect of the Therapeutic

Suitable in vitro or in vivo assays can be performed to determine the effect of a specific therapeutic and whether its administration is indicated for treatment of the affected tissue.

In various specific embodiments, in vitro assays may be performed with representative cells of the type(s) involved in the patient's disorder, to determine if a given therapeutic exerts the desired effect upon the cell type(s). Modalities for use in therapy may be tested in suitable animal model systems including, but not limited to rats, mice, chicken, cows, monkeys, rabbits, and the like, prior to testing in human subjects.

Similarly, for in vivo testing, any of the animal model systems known in the art may be used prior to administration to human subjects.

5.10.5 Prophylactic and Therapeutic Uses of the Compositions of the Invention

MO-1 nucleic acids and proteins are useful in potential prophylactic and therapeutic applications implicated in a variety of disorders including, but not limited to, diabetes, type II diabetes, obesity, hyperglycemia, insulin resistance, hyperinsulinemia, hypercholesterolemia, hypertension, hyperlipoproteinemia, hyperlipidemia, hypertriglylceridemia and dyslipidemia and the like.

As an example, a cDNA encoding MO-1 may be useful in gene therapy, and the protein may be useful when administered to a subject in need thereof. In some embodiments, the MO-1 polypeptide is administered in a form that permits entry of the MO-1 polypeptide into a cell, e.g., an adipocyte. Formulations for accomplishing this are described above. By way of non-limiting example, the compositions of the invention may have efficacy for treatment of patients suffering from obesity. In other embodiments, the compositions of the invention may be useful in methods of increasing the weight of a subject, e.g., a subject in need of increased body mass. In some embodiments, the compositions may be used to treat, for example, cachexia.

MO-1 nucleic acids, or fragments thereof, may also be useful in diagnostic applications, wherein the presence or amount of the nucleic acid or the protein is to be assessed. A further use could be as an anti-bacterial molecule (i.e., some peptides have been found to possess anti-bacterial properties). These materials are further useful in the generation of Abs that immunospecifically bind to the novel substances of the invention for use in therapeutic or diagnostic methods.

### 6. EXAMPLES

The invention is illustrated by the following examples which are not intended to be limiting in any way.

### 6.1 Example 1

### Identification and Mapping of MO-1

This example describes mapping and sequencing of a gene associated with morbid obesity, type II diabetes heart disease, and hypertension.

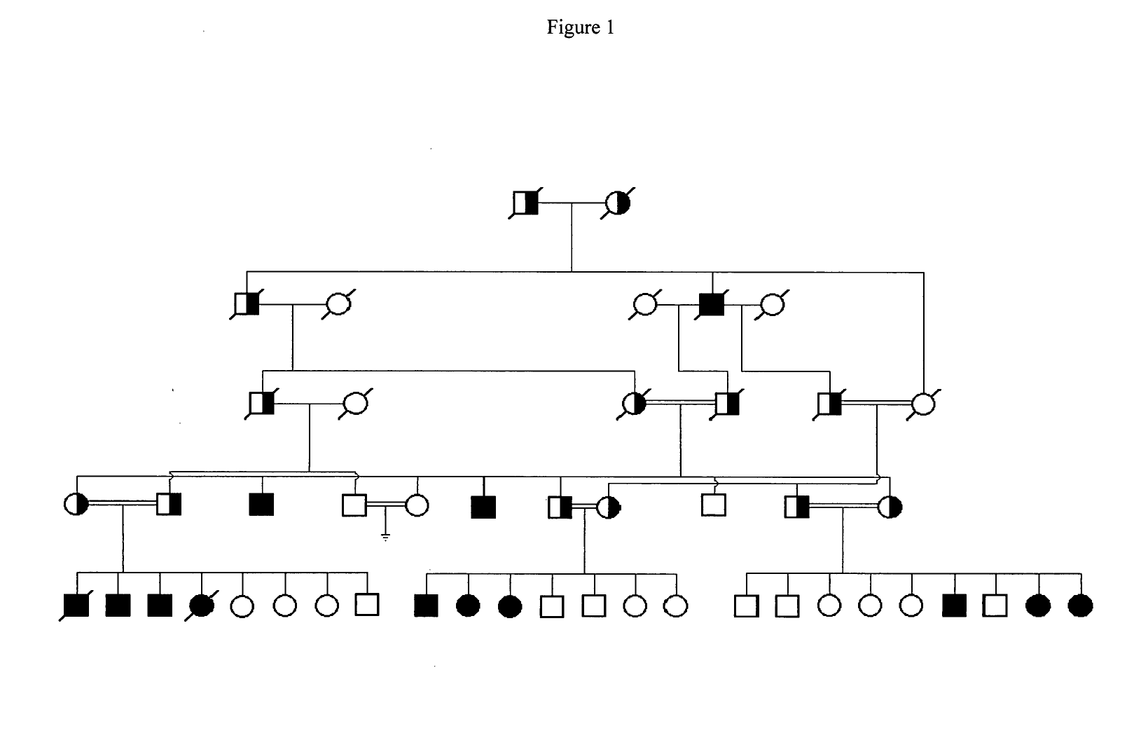

A large, consanguineous multigenerational family with morbid obesity, type II diabetes, heart disease and hypertension was identified; FIG. 1 shows the lineage of this family. Detailed clinical data were obtained for all kindred members, including all 10 living affected individuals, shown in Table 3, below. Family members were known by history to have normal gestational birth weights but by age 2-3, affected children had increased BMIs. Affected adults had average BMIs ˜45. Three of 12 affected individuals had mild mental retardation but all had normal sexual development. In addition, three individuals had died of coronary artery disease/myocardial infarction, three of twelve affected family members were type II diabetics, and eleven of twelve had hypertension. Four of six individuals had abnormal lipid profiles including elevated triglyerides and cholesterol.

The obesity phenotype was inherited as an autosomal recessive trait. Therefore, a positional-cloning strategy was used to identify the causative gene by identifying homozygous-by-descent regions in affected individuals. Microsatellite markers from the Human Screening Panel, version 9.0 (Research Genetics), were used for the genome wide scan. Additional markers (Research Genetics; Integrated DNA Technologies) were obtained to refine the critical region. The model assumed the trait to be a highly penetrant, autosomal recessive disorder with a disease allele frequency of 1:10,000.

A LOD score of 9.7 (Φ=0) was observed within a 5.5 Mb region on the telomeric end of chromosome 3q29 between markers D3S2418-D3S3550. Additional markers in this region were analyzed and the multipoint lod scores for the identified region of homozygosity were determined. Heterozygosity and haplotype analysis narrowed this initial region to the 1.6 Mb critical region between markers centromeric marker D3S2306 and telomeric marker D3S3550.

Inspection of the genes mapped to the region revealed a number of candidate genes based on their known or probable functions and expression pattern. A number of candidate genes including BDH1, (R)-3-hydroxybutyrate dehydrogenase (EC 1.1.1.30), which is required for the interconversion of the two major ketone bodies produced during fatty acid catabolism, DLG1, involved in cell proliferation and signaling, and PAK2, a member of the p21 activated kinase family that link Rho GTPases to cytoskeleton reorganization and nuclear signaling. No homozygous mutations in any of the candidate genes in the region which segregated appropriately within the family were identified.

However, an EST sequence, FLJ25426 or C3orf34, was identified that encodes a 167 amino acid protein with a calculated MW of 19.6 kD, i.e., MO-1 (SEQ ID NO:1), and which shared some sequence homology to phosphoenolpyruvate carboxylase and carboxykinase. PCR primers were then designed to all three 3 exons and flanking intronic and untranslated sequences, including the two coding exons, and directly sequenced all affected family members. All affected individuals were homoallelic for the same nonsense mutation, a C→T transition in codon 82 of exon 2, which predicts premature truncation of the protein (R82X) and loss of greater than half of the protein sequence. This mutated form of MO-1 polypeptide is also contemplated to be a part of the present invention. This nucleotide substitution segregated appropriately within the family; unaffected parents were heteroallelic for the mutation while unaffected siblings and relatives were either heteroallelic or had the wildtype sequence. In addition, the mutation was not present in greater than 500 chromosomes from 250 unaffected, unrelated control individuals.

### 6.2 Example 2

### Generation of a MO-1-Specific Monoclonal Antibody

This example describes the generation and isolation of a monoclonal antibody preparation that specifically binds the MO-1 polypeptide.

A monoclonal antibody was generated as follows. Mice were immunized with Keyhole Limplet Hemocyanin (KLH) conjugated with a peptide selected from the MO-1 polypeptide sequence (CTRAAEQLKNNPRH; SEQ ID NO.:11). Two booster immunizations were subsequently administered. Post-immunization serum was then used in an ELISA assay against free peptide to assess the monoclonal antibody response using conventional techniques.

Pre-immune serum was used as negative control (data not shown). Data from the ELISA assay is shown as Table 4, below.

TABLE 4

| | Animal Number | | | | |
|---|---|---|---|---|---|
| Dilution | 1 | 2 | 3 | 4 | 5 |
| 1:1000 | 2.431 | 1.847 | 2.233 | 1.065 | 1.015 |
| 1:3000 | 1.553 | 1.296 | 1.326 | 0.525 | 0.785 |
| 1:9000 | 0.915 | 0.713 | 0.679 | 0.254 | 0.584 |
| 1:2,7000 | 0.245 | 0.311 | 0.477 | 0.202 | 0.285 |
| 1:8,1000 | 0.169 | 0.266 | 0.193 | 0.173 | 0.157 |
| 1:24,3000 | 0.119 | 0.182 | 0.161 | 0.123 | 0.152 |
| Blank | 0.106 | 0.111 | 0.123 | 0.117 | 0.111 |
| Titer | >27000 | >81000 | >27000 | >9000 | >27000 |

In Table 4, the titer with the highest dilution wherein Signal/Noise (Sample/Blank) >=2.1 is shown in bold.

### 6.3 Example 3

### Generation of a MO-1-Specific Polyclonal Antibody Preparation

This example describes the generation and isolation of a MO-1 polypeptide-specific polyclonal antibody preparation.

A MO-1 peptide comprising amino acids 133-153 of the MO-1 protein sequence (DPNFVYDIEVEFPQDDQLQSC; SEQ ID NO:21) was synthesized and conjugated with either KLH or ovalbumin as adjuvants and injected into rabbits. After confirming high titers by ELISA, the serum was then tested for its ability to detect V5-tagged-MO-1 by Western blotting techniques. Confirmatory blots were done using the V5 antibody. RbtA1783 from both the crude serum (1:1500 dil) and following affinity purification (1:200) reveals the presence of a highly prevalent band corresponding to the predicted size of MO-1/V5. The same extract probed with an antibody recognizing the V5 tag also identified a similar band corresponding to the predicted size of MO-1/V5.

### 6.4 Example 4

### Generation of a MO-1-Knockout Mouse

This example describes the generation and isolation of a MO-1 knockout mouse.

First, a knockout vector was constructed and identified with a PCR-based screen. Ten micrograms of the targeting vector was linearized by NotI and then transfected by electroporation of iTL1 IC1 C57BL/6 embryonic stem cells. After selection with G418 antibiotic, surviving clones were expanded for PCR analysis to identify recombinant ES clones.

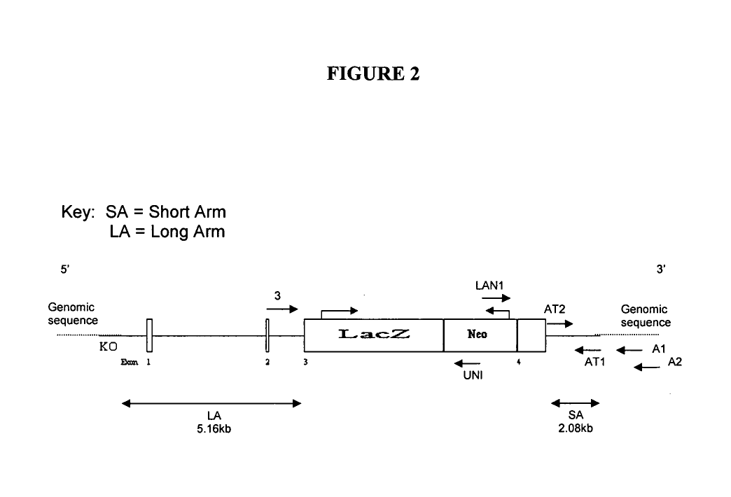

Screening primers A1 and A2 were designed downstream of the short homology arm (SA) outside the 3′ region used to generate the targeting construct, shown diagramatically in FIG. 2. PCR reactions using A1 or A2 with the LAN1 primer (located within the Neo cassette) amplify 2.4 and 2.5 kb fragments, respectively. The control PCR reaction was performed using the internal targeting vector primers AT1 and AT2, which are located at the 3′ and 5′ ends, respectively, of the SA. This amplifies a product 1.3 kb in size.

Individual clones from positive pooled samples were then screened using the A2 and LAN1 primers. Positive recombinant clones were identified by a 2.5 kb PCR fragment. Next, positive SA PCR clones were sequenced for integration using the OUT1 primer. Clones 133, 134, 151, 152, 154, 171, 172, 173, 174, 211, 214, 241, 243 and 244 were selected and tested for cassette integration.

Confirmation of cassette integration within the long homology arm was performed by PCR using the 3 and UNI primers. Sequencing was performed on purified LA PCR DNA to confirm presence of the cassette junctions using the 3 and N7 primers. Clones 133, 151, 154, 172 and 174 were selected for expansion and reconfirmation using the same methods described above. Clones 133, 151, 154, 172 and 174 were successfully reconfirmed and were determined to be suitable for injection

The engineered embryonic stem cells are then inserted into a mouse blastocyst which are then implanted into the uterus of female mice, to complete the pregnancy. The blastocysts then contain two types of stem cells: the ones from the contributing mouse and the newly engineered ones. A color selection based on coat color is used to discriminate between the two donors (for example, black and white fur). The newborn mice are therefore chimeras: parts of their bodies result from the original stem cells, other parts result from the engineered stem cells and as such, their furs will show patches of the different colors.

Newborn mice with the newly engineered knockout sequence incorporated into the germ cells (egg or sperm cells) are then crossed with others of the relevant genetic background for offspring that are “pure”. These mice now contain one functional copy and one “deleted” copy of the gene and are further inbred to produce mice that carry no functional copy of the original gene (i.e. are homozygous for the knockout) and can be detected by PCR or Southern blot.

### 6.5 Example 5

### Generation of a MO-1-Transgenic Mouse

This example describes the generation and isolation of a transgenic mouse expressing the human MO-1 polypeptide.

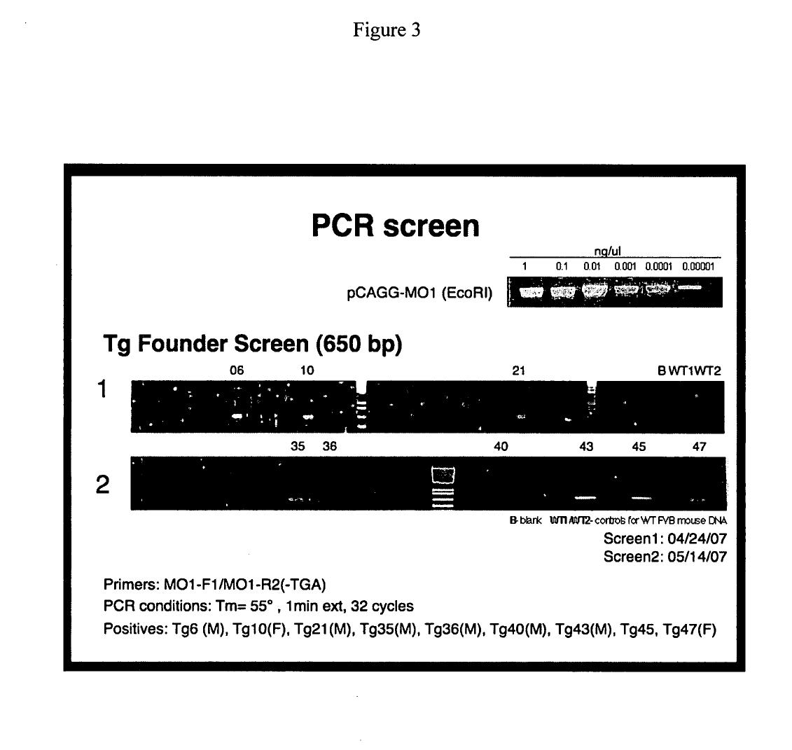

Generation of Mice. To generate the targeting construct, the human MO-1 genomic sequence was amplified using total genomic DNA isolated from the human Hep3B cell line. The primers MO-1-FL-F1, which sits on the 5′UTR region on exon 2, and MO-1-FL-R2, which sits on the 3′UTR and includes the native STOP codon, were used to amplify the sequence to be inserted into the pCAGG multiple cloning site (MCS) which utilizes the chicken beta-actin promoter to drive ubiquitous expression of the gene of interest. The MO-1 sequence was introduced into the vector using the vector's EcoRI site. Upon purification, the DNA was microinjected into FVB mouse ES cells to allow for genomic integration of the exogenous DNA. The cells were then transplanted into a pseudopregnant female mouse for normal gestation. Pups were then screened for the presence of the transgene by Southern blotting. MO-1 probe was generated by digesting the transgenic construct with EcoRI and purifying the 650 bp product. A PCR-based screen was then developed using the MO-1-FL-F1/MO-1-FL-R2 in order to facilitate ease of screening subsequent generations (FIG. 3). Pure lines were generated by crossing transgenic founders with wild type FVB females, thus generating F1 transgenic pups.

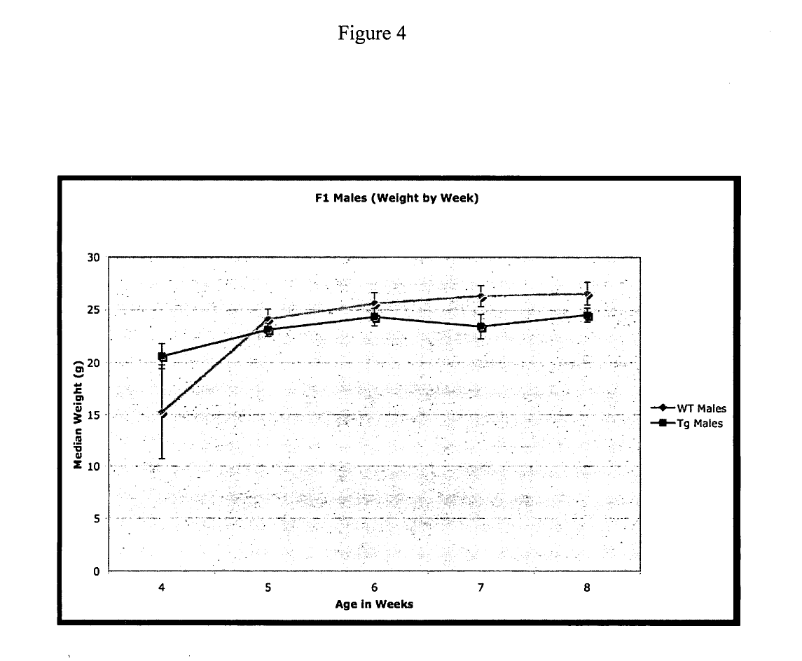

Characterization of Mice. A. Weight. Two independent MO-1 transgenic lines were compared to wild type littermates to assess differences in weight. As shown in FIG. 4 below, overexpression of MO-1 is associated with a more lean body mass.

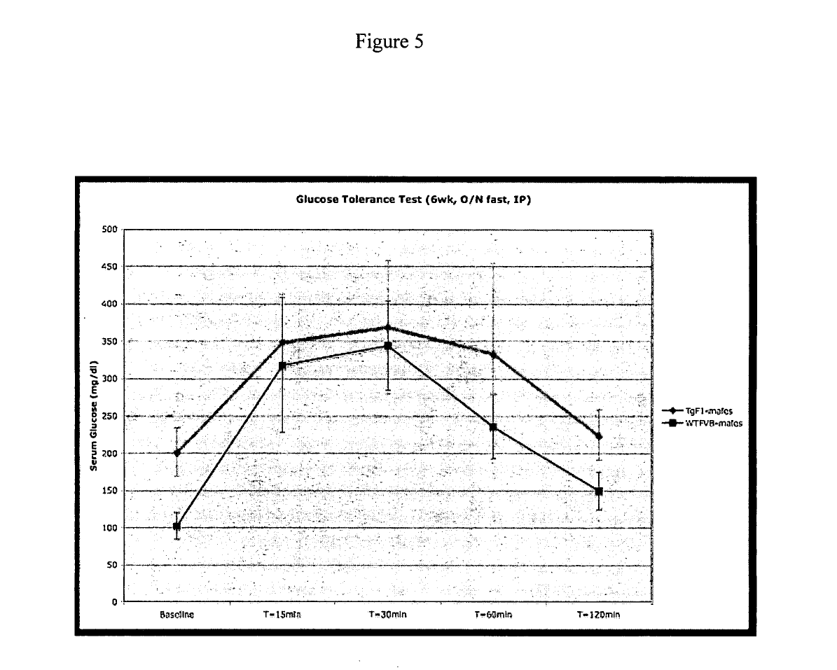

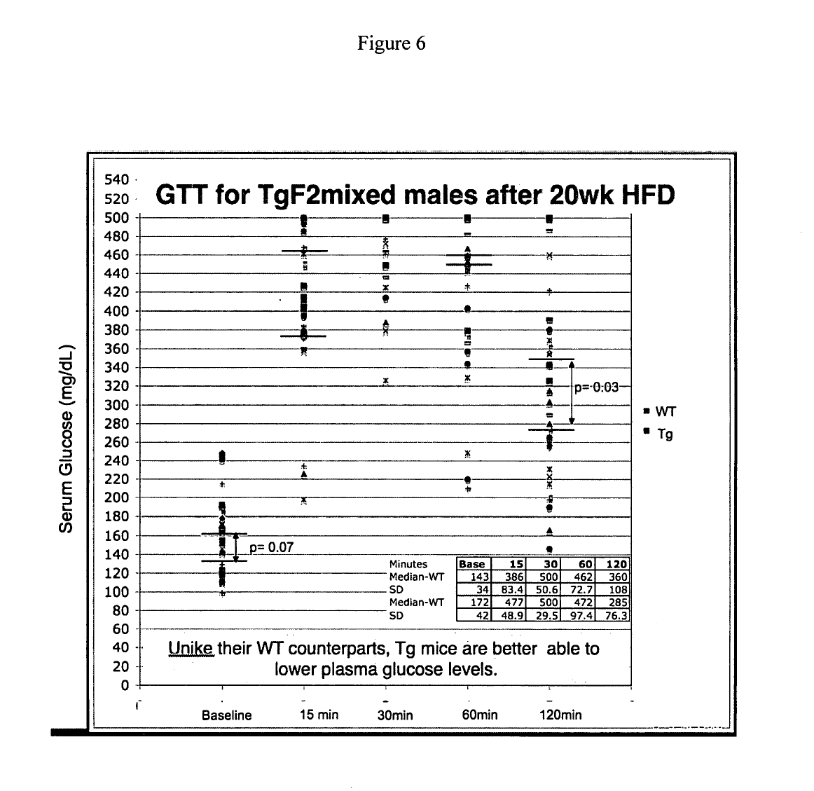

B. Serum Glucose and Glucose Tolerance Testing. Glucose tolerance testing was performed on transgenic and wild type littermates at two distinct developmental timepoints and under different dietary conditions. First, on animals at 6 weeks of age. Second, on animals at 27 weeks of age after having been placed on a high-fat (60% fat) for 20-weeks. In both cases, and counterintuitively but consistent with the in vitro data, the transgenic mice had elevated serum glucose levels following an overnight fast (FIGS. 5 and 6). The transgenic mice maintained consistently elevated glucose levels when compared to their wild type littermates. Of note, in the HF diet experiments, MO-1 transgenic mice, despite starting at higher initial levels (trend/p=0.07), displayed a significantly improved response to lowering serum glucose at 2 hours (˜80 mg/dL; p<0.03). Briefly, for both experiments, after a 16-hour overnight fast, mice were injected intraperitoneally with a bolus of glucose (D-50) at a dose of 1 g glucose/kg total body weight. Plasma glucose was then measured at 15, 30, 60, and 120 minutes post injection using a glucomoter (Freestyle Flash Glucose Meter, Abbott Diagnostics).

### 6.6 Example 6

### Identification and Characterization of Single Nucleotide Polymorphisms Associated with MO-1 Alleles

This example describes the identification and characterization of Single Nucleotide Polymorphisms (SNPs) associated with MO-1 alleles.

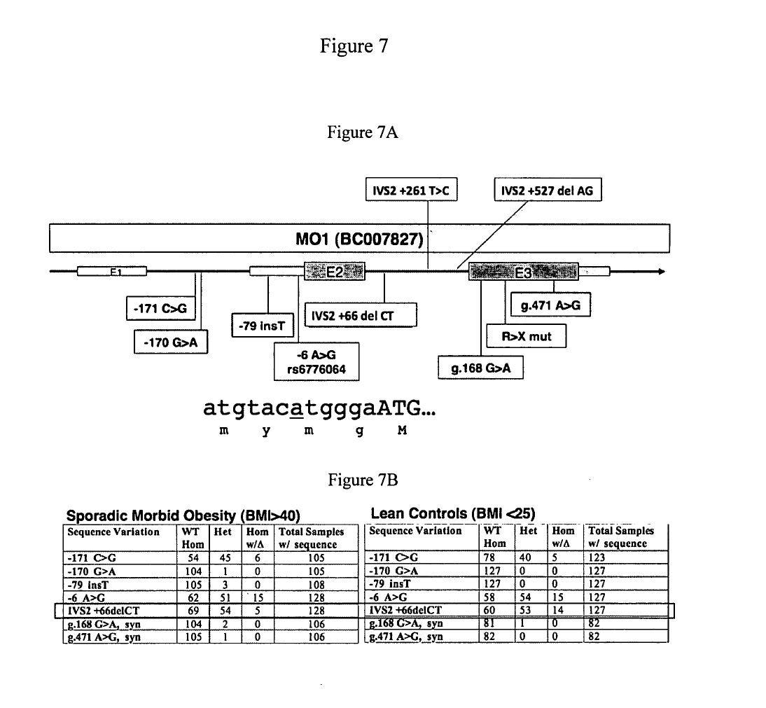

Over 500 individuals of primarily European ancestry and separated according to body mass index (BMI) were directly sequenced at the MO-1 gene locus on chromosome 3q29 to identify single nucleotide polymorphisms (SNPs), useful in diagnostic/prognostic assays. In the figure, the MO-1 gene locus is shown with the presumed MO-1 start site in capital letters below the diagram. Using the ATG as the start site reference (i.e. position +1), individual SNPs and their relative positions are shown as FIG. 7A. IVS=intervening sequence/intronic sequence.

As an example of diagnostic/prognostic utility, SNP IVS2 +66 del CT was used in an association study comparing lean (BMI <25) and obese individuals (>40) in a sample set of approximately 300 individuals in total. As shown in FIG. 7B, the IVS2 +66 del CT SNP was present nearly 3 times more frequently in the homozygous state in lean individuals (Chi-square=4.68, p<0.05); suggestive of a protective effect on weight gain in this BMI range.

### 6.7 Example 7

### Assays Testing Effects of MO-1 Overexpression and Silencing

This example describes assays assessing the effects of MO-1 overexpression and silencing.

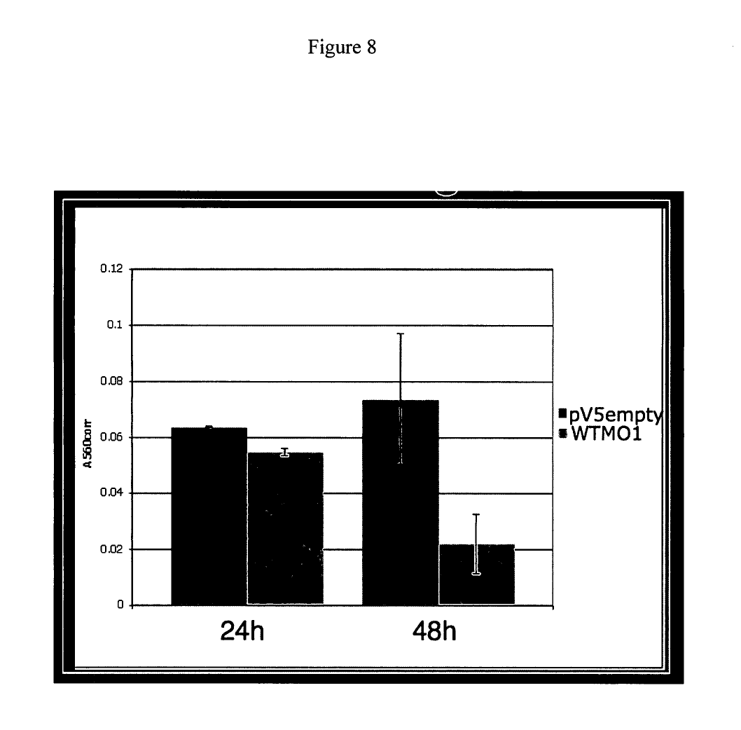

Increased Expression Results in Decreased Intracellular Glucose Levels. In the first experiment, Hep3B cells, a liver-derived human cell line with gluconeogenic capacity were transfected with either control (empty) or MO-1 overexpressing vectors. The cells were maintained in serum-free, low glucose media (low energy condition) and supplemented with an excess of pyruvate and lactate (known substrates of gluconeogenesis). As shown in FIG. 8, overexpression of MO-1 results in decreased (˜70%) intracellular glucose levels.

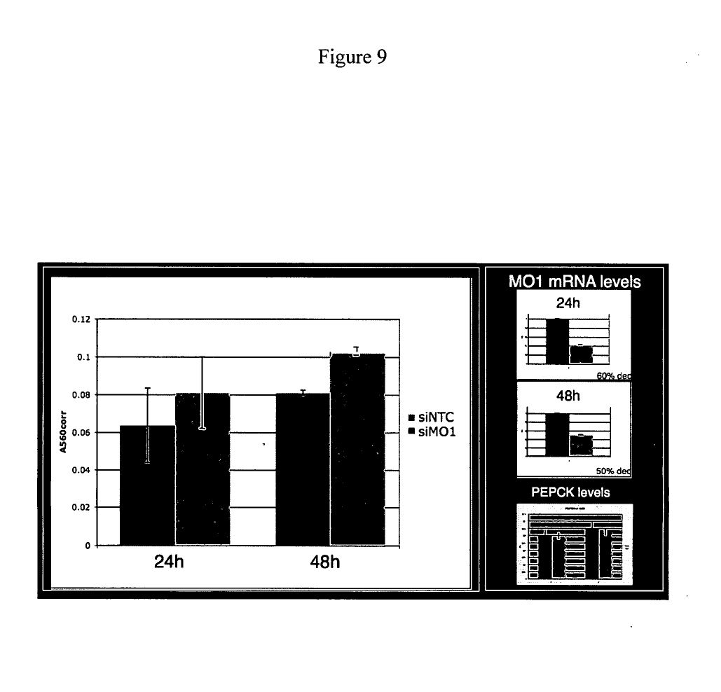

Decreased Expression Results in Increased Intracellular Glucose Levels. In this experiment, siRNA directed against MO-1 was used to silence the expression of endogenous MO-1 in Hep3B cells. As shown in FIG. 9, silencing of MO-1 (˜50% at 48 hours) resulted in increased intracellular glucose levels (˜25%). The specificity of the siRNA towards MO-1 is shown by the fact that PEPCK RNA levels are unchanged following MO-1 targeted silencing.

Effects of MO-1 silencing on preadipocyte, NIH 3t3L, differentiation. In this experiment, NIH 3T3 L1 cells were plated at low density. Day 0 cells were stained and collected for RNA. Remaining cells were transfected with siNTC (control) or siMO1 at Day 0 and induced 7 hrs later using 0.5 mM IBMX/1 uM Dexamethasone/1 uM Insulin. Cells were kept in induction media for 3 days. Day 3 cells were stained and collected for RNA. Remaining cells were re-transfected with siNTC or siMO1 and media was replaced after 7 hrs with maintenance media containing 1 uM Insulin. Cells were left in this media for 3 days.

At Day 6, remaining cells were again re-transfected with siNTC or siMO1 and media was changed with fresh maintenance media (contains 1 uM Insulin) 7 hrs later. •Day 8 cells were stained and collected for RNA Successive transfections were done to maintain knock-down of MO-1 throughout the course of the experiment.

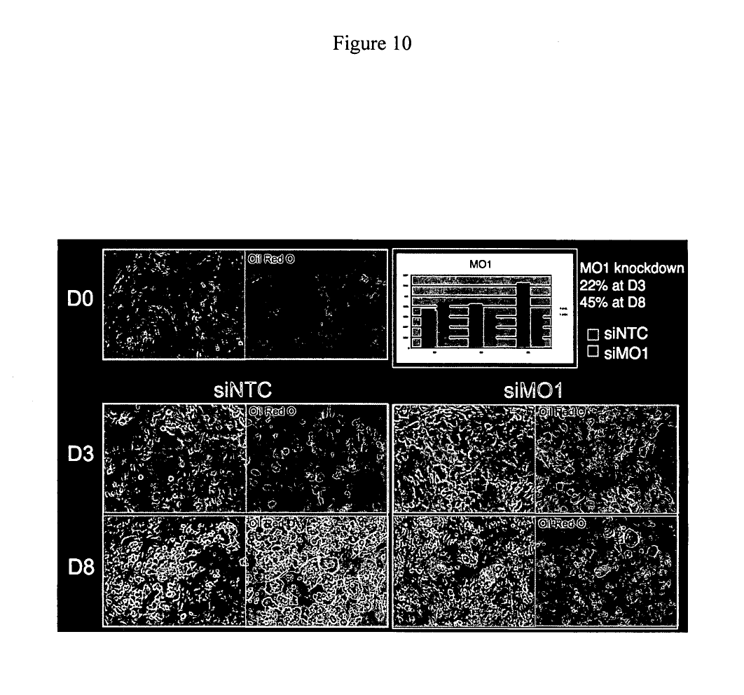

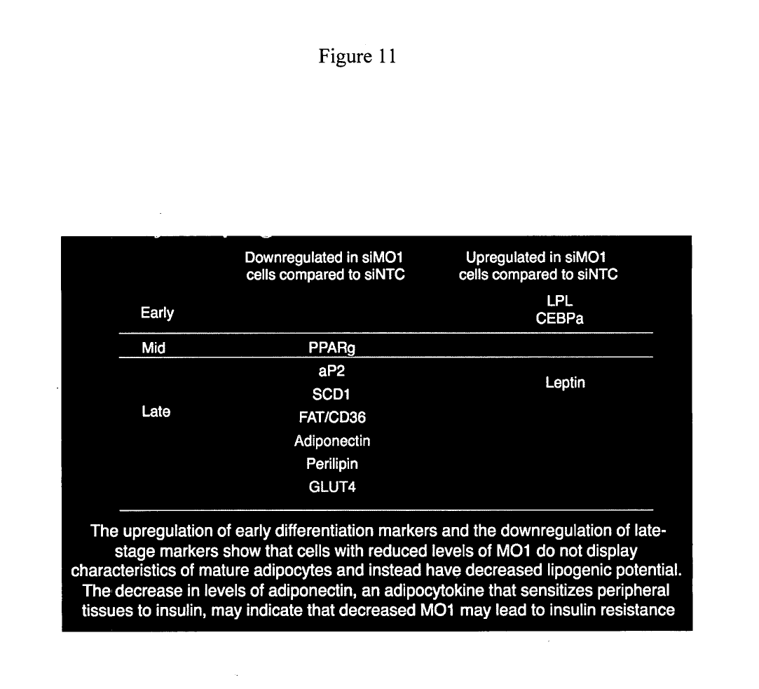

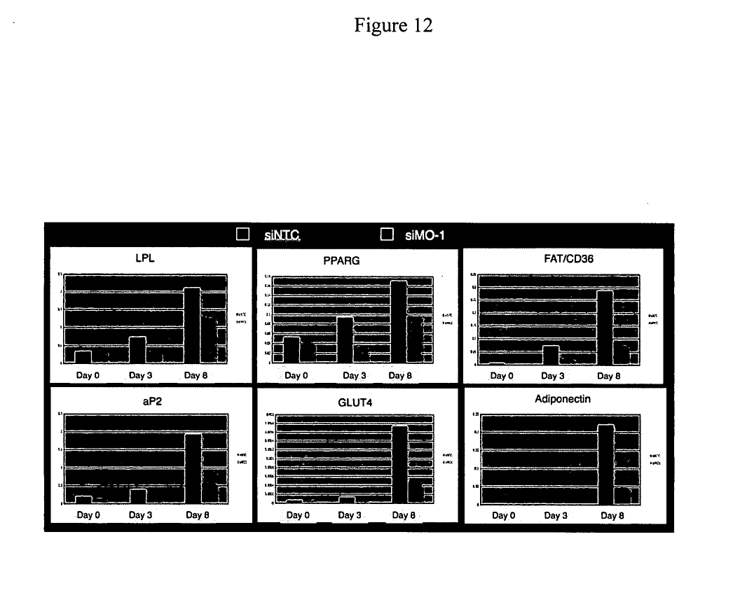

MO-1 expression decrease resulted in delayed adipocyte differentiation/adipogenesis and reduction of intracellular lipid accumulation (FIG. 10). In addition, decreased levels of MO-1 resulted in altered expression of key adipogenic markers of differentiation throughout different stages of adipocyte development (FIGS. 11 and 12). Shown are quantitative real-time PCR data of a number of these key regulatory genes. These results suggest that alterations in expression levels of MO1 can affect a number of critical genes and pathways involved in adipocyte differentiation and adipogenesis

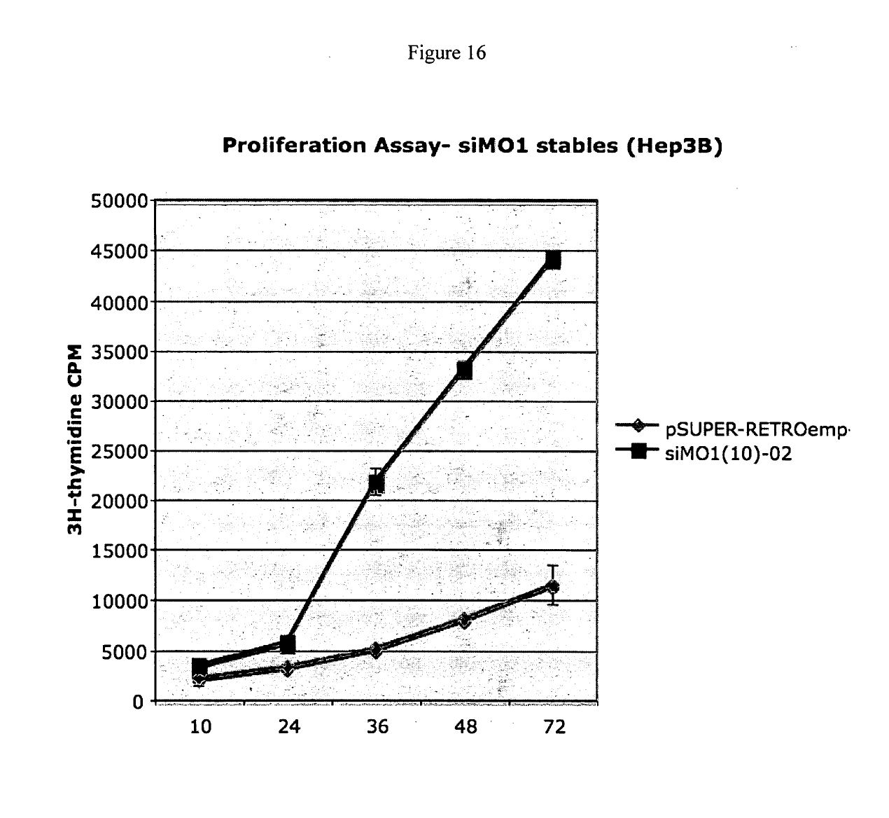

In another experiment, Hep3B cells were infected with a retrovirus expressing an siRNA targeting MO-1 and cells stably expressing the siRNA were selected. This resulted in long-term reduction in MO-1 expression. Among other results, ablation of MO-1 expression by approximately ˜90% resulted in increased cell proliferation (FIG. 16).

### 6.8 Example 8

### Assessing Expression Patterns and Localization of MO-1 Expression

This example describes the results of experiments designed to assess expression patterns of MO-1 in tissues and within cells.

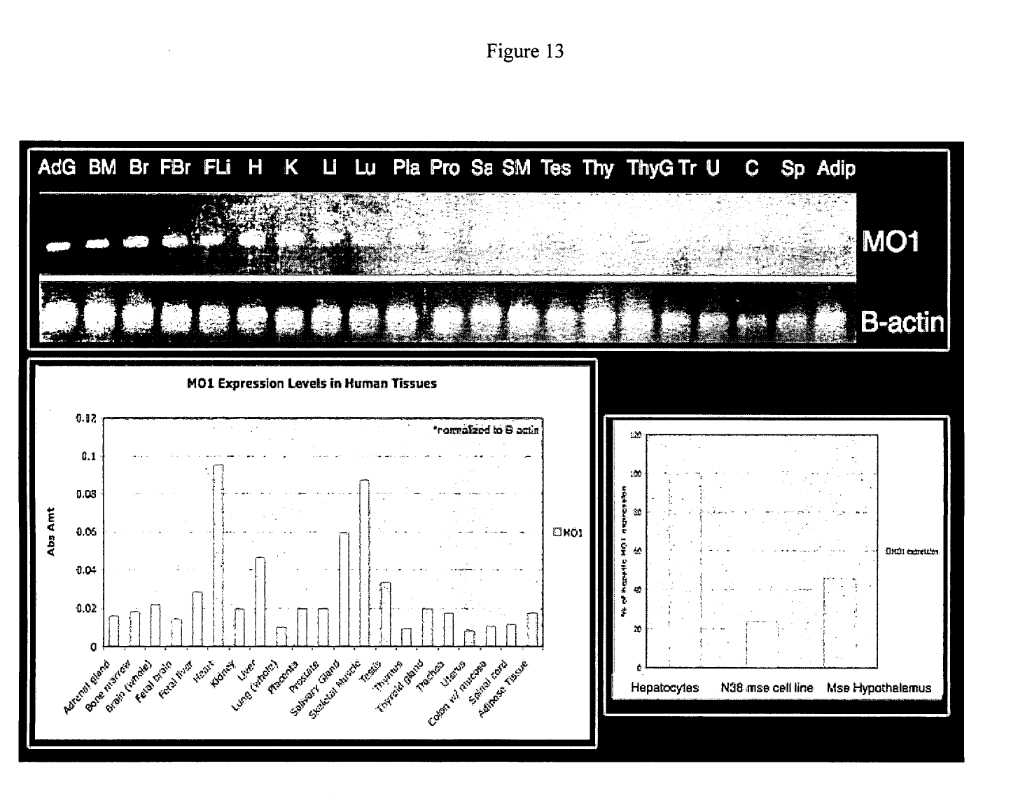

First, RNA was isolated from various human tissues and assayed by RT-PCR for expression of MO-1 mRNA in the tissues. Results from this assay are shown as FIG. 13. As indicated in FIG. 13, MO-1 is expressed in multiple tissues, including adipocytes, liver, muscle and hypothalamus.

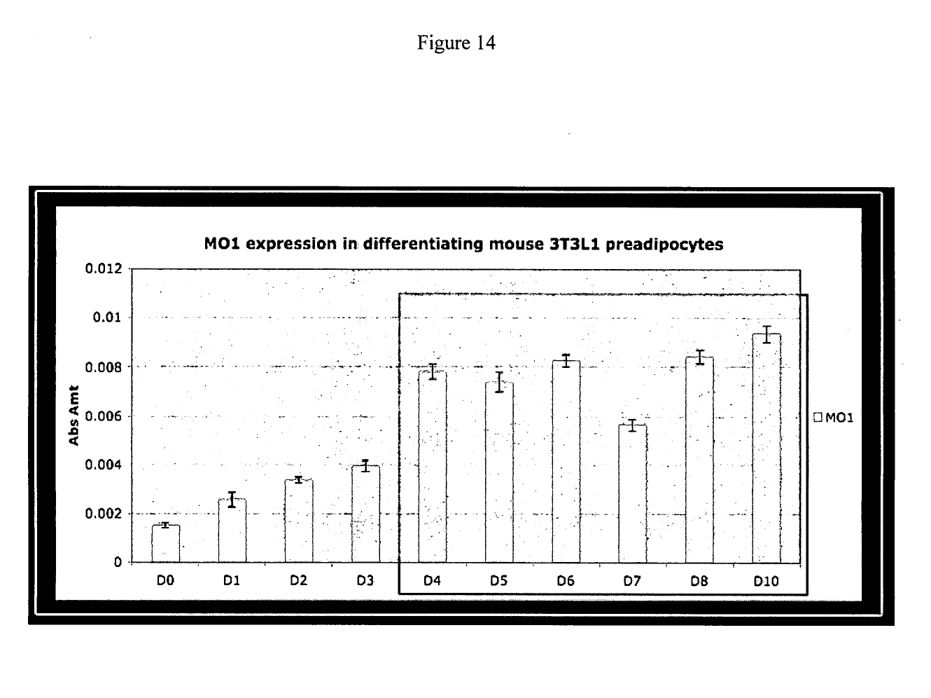

Next, NIH 3T3 L1 cells were induced to differentiate as described in Example 7, above, and RNA was extracted once daily for 10 days. MO-1 RNA levels were determined by qRT-PCR. Results are shown in FIG. 14. Interestingly, increased levels of MO-1 coincide with onset of lipogenesis.

Finally, tagged MO-1 protein was expressed in Hep3B cell lines and the pattern of expression was assessed by immunofluorescence. MO-1 protein was expressed in the cytoplasm and localized to the mitochondria.

### 6.9 Example 9

### Assessing MO-1 Protein-Protein Interactions

This example describes the results of experiments designed to assess interactions MO-1 with other proteins.

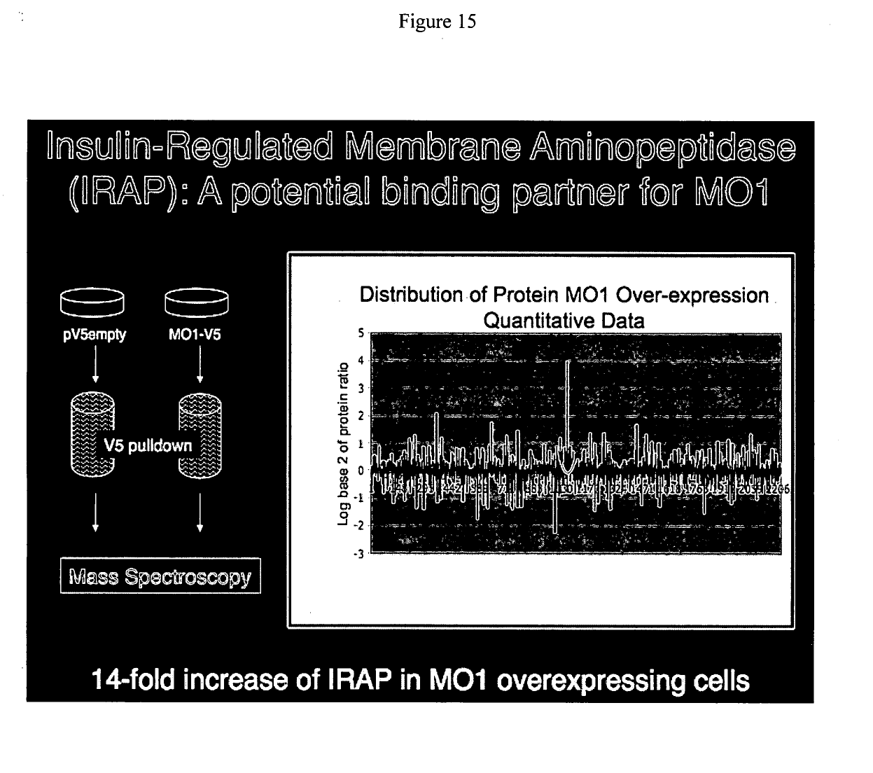

Two complementary methods were used to identify potential MO1 protein binding partners. First, a mass spectroscopy approach identifying the differences between MO1-V5 tagged and empty-V5 overexpressed proteins was pursued. The schema is shown in FIG. 15. Using this approach in Hep3B cells, 1 predominant protein was identified: IRAP.

This interaction is believed to be of particular relevance since IRAP:
is a member of the Zn-dependent family of membrane aminopeptidases
contains a single TM domain
localizes to GLUT4-containing intracellular vesicles under basal conditions in response to insulin:
redistributes to the cell surface along with GLUT4 to facilitate glucose disposal;
cleaves extracellular peptide hormone substrates (eg. vasopressin)
exhibits impaired translocation to cell surface in muscle, liver and adipose tissue in type II diabetes; and
results in 50-80% decreased GLUT4 in muscle, liver and adipose tissue of IRAP −/− mice due to degradation as a result of impaired sorting and trafficking.

Next, the yeast two hybrid system was used to screen of a human liver library. Several binding genes were identified:

SCP2: the SCP2 gene encodes two proteins: sterol carrier protein X (SCPx) and sterol carrier protein 2 (SCP2), as a result of transcription initiation from two independently regulated promoters. The transcript initiated from the proximal promoter encodes the longer SCPx protein, and the transcript initiated from the distal promoter encodes the shorter SCP2 protein. The two proteins share a common C-terminus. SCPx is a peroxisome-associated thiolase that is involved in the oxidation of branched chain fatty acids. SCP2 protein is an intracellular lipid transfer protein. This gene is highly expressed in organs involved in lipid metabolism. Of note, SCP2 plays an important role in intracellular movement of cholesterol and possibly other lipids. SCP2 is believed to facilitate the transport of cholesterol to mitochondria, where the first committed step in steroidogenesis takes place.

CYP2B6 (GID: 82583665), encodes a member of the cytochrome P450 superfamily of enzymes. As a family, cytochrome P450 proteins are monooxygenases which catalyze many reactions involved in drug metabolism and synthesis of cholesterol, steroids and other lipids.

MTO1-like (GID: 17149038), a mitochondrial protein ubiquitously expressed in various tissues, but with markedly elevated expression in tissues of high metabolic rates.

In addition, three hypothetical proteins were identified, and thus they themselves may play a previously unrecognized role in metabolism: GID: 14578073, GID: 158819059, and GID: 21212494.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

TABLE 3

| | | Gest. | | Birth | | | Recent | | | | | |
|---|---|---|---|---|---|---|---|---|---|---|---|---|
| | | Age | Date of | Weight | Breast | Obesity | Weight | Height | | Development* | | |
| Patient | Gender | (weeks) | Birth | (KG) | Feeding | Onset | (Kg) | (cm) | BMI | Mental | Sexual | HTN |
| PATIENT 1 | M | 38 | 1961 | 3.950 | Yes | 2-3 yrs | 137 | 175 | 44.7 | N | N.# | Yes** |
| PATIENT 2 | M | 39 | 1953 | 4.050 | Yes | 2-3 yrs | 145 | 175 | 47.3 | N. | N.# | Yes** |
| PATIENT 3 | F | 40 | 1976 | 3.35 | Yes | 2-3 yrs | 147 | 170 | 50.9 | N. | N. | No |
| PATIENT 4 | F | 40 | 1977 | 3.3 | Yes | 3 yrs | 145 | 169 | 51.4 | N. | N. | Yes** |
| PATIENT 5 | M | 33 | 1972 | 1.5 | Yes | 4 yrs | 122 | 182 | 36.8 | Mild MR | N. | Yes** |
| PATIENT 6 | F | 40 | 1974 | 4 | Yes | 1 yr | 121 | 165 | 44.4 | N. | N. | Yes** |
| PATIENT 7 | F | 39 | 1977 | 3.8 | Yes | 1 yr | 134 | 165 | 49.2 | N. | N. | Yes** |
| PATIENT 8 | M | 39 | 1969 | 3.95 | Yes | 2-3 yrs | 115 | 165 | 42.2 | Mild MR | N. | Yes** |
| PATIENT 9 | M | 40 | 1965 | 4 | Yes | 3 yrs | 135 | 165 | 49.6 | N. | N.# | Yes** |
| PATIENT 10 | M | 39 | 1980 | 3.9 | Yes | | 115 | 183 | 34.3 | Mild MR | N. | Yes** |
| PATIENT 11 | M | 40 | Died 25 yrs | 3.9 | Yes | 2-3 yrs | 130 | 165 | 47.8 | N. | N. | Yes** |
| PATIENT 12 | F | 40 | Died 16 yrs | 3.95 | Yes | 3 yrs | 120 | 160 | 46.9 | N. | N. | Yes** |

| | | | | | | | | | | |
|---|---|---|---|---|---|---|---|---|---|---|
| Average | Term | 3.83 | Yes | 2-3 yrs | 130.5 | 169.9 | 45.45 | 3/12 | N. | 10/11 |
| | | | | | | | | have | | Have |
| | | | | | | | | MR- | | HTN |
| | | | | | | | | mild | | |

Primers for amplifying and sequencing genomic MO1

| | |
|---|---|
| MO1.1R | CTTGTTAGGAAGCCCCACAG |
| MO1.2F | CGCAAGGATGACACACAAAT |
| MO1.2R | GGAAACTGAAGCTCACTGAGAGTA |
| MO1.3F | AATATTTTCAGGGTTCAGAGTTTTT |
| MO1.3R | TTGAAAAGTAACATGGTCAATCC |
| MO1-EX3R | GGATTGACCATGTTACTTTTCAA |
| MO1-FL-F1 | TTC ATC AGA TTT CCT CTG ACT TAG |
| | CCG G |
| MO1-FL-R1(-TGA) | GAG TGT TTG GTA TGA GAA CTC ATC |
| | AGC TG |
| MO1-FL- | GTA ACA TGG TCA ATC CCC TAT AAC |
| R2(TGA + 93) | CCA AC |

Primers for genotyping MO1 Transgenic Mice

| | |
|---|---|
| MO1-FL-F1 | TTC ATC AGA TTT CCT CTG ACT TAG |
| | CCG G |
| MO1-FL- | GTA ACA TGG TCA ATC CCC TAT AAC |
| R2(TGA + 93) | CCA AC |

MO1 Primers to amplify Full-length cDNA

| | |
|---|---|
| MO1-FL-F1 | TTC ATC AGA TTT CCT CTG ACT TAG |
| | CCG G |
| MO1-FL-R1(-TGA) | GAG TGT TTG GTA TGA GAA CTC ATC |
| | AGC TG |
| MO1-FL- | GTA ACA TGG TCA ATC CCC TAT AAC |
| R2(TGA + 93) | CCA AC |

MO1 Primers to amplify R > X cDNA
(used Rev right after mut to simulate the
truncated protein since TGA cannot be used because
of 3′ tag (V5)

| | |
|---|---|
| MO1-R > X trunc- | CTG CCC CGA CAA GTA ACC TCG TAA |
| Rev1 | |
| MO1-R > X trunc- | CTG AAT AGC TTC TCT AGC TGC CTC |
| Rev2 | AGG |

MO1 human Realtime Primers

| | |
|---|---|
| MO1-RT-F1 (+22) | GGG ATT AGG TTT GAG CCT CCA GC |
| MO1-RT-R1(+196) | GAA TAG CTT CTC TAG CTG CCT CAG GG |
| MO1-RT-F2(+70) | GAA ATC AAG GGG AAA ATT CGC CAG CG |
| MO1-RT-R2(+246) | GTT TCT GCC AGA CTC TGC CCC |
| MO1-RT-F3 | GCA CTG CCA AGA AAT GTG GGA TTA GG |
| MO1-RT-R3 | CTT TAA TTG TTC AGC AGC TCT GGT GC |

MO1 mouse Realtime Primers

| | |
|---|---|
| MO1-Mse-RT-F1 | GAG TTA GGT TCC AGC CTC CAG C |
| MO1-Mse-RT-R1 | CAG GTA ACT CTT GTG CCG TGG G |
| MO1-Mse-RT-F2 | GAA TGA AAC CGA AGG AAA GAG CCG C |
| MO1-Mse-RT-R2 | CGC AAA AAA ACA AAC AGC TTC TCC |
| | AGC |

For pBABE-MO1-puro construct

| | |
|---|---|
| MO1pBABE-EcoRI-F | TTG CCC TTG AAT TCA GAT TTC CTC |
| | TGA CTT ACC |
| MO1-pBABE + V5- | GGC TGC TCG ACG GGT TTA AAC TCA |
| SalI-R | ATG G |

For MO1 SNaPshot screen

| | |
|---|---|
| MO1-SNaP-delCT- | CTC TAA ATA CAG GTT CAC ATC ATG |
| 27F | ACT |
| MO1-SNaP-delCT- | GTG TCA CAC ATC CAG TCC CTG ACA G |
| 25R | |

For synthetic siMO1 generation for cloning into
pSUPER-RETRO-puro

| | |
|---|---|
| siMO1(10)-Fwd | gatccccAAGAATAATCCGCGACACATTttcaag |
| | agaAAT GTG TCG CGG ATT ATT CTTtttt |
| | tggaaa |
| siMO1(10)-Rev | agcttttccaaaaaAAGAATAATCGGCGAGACAT |
| | TtctcttgaaAAT GTG TCG CGG ATT ATT |
| | CTTggg |

What is claimed is:

1. An isolated nucleic acid encoding a polypeptide comprising an amino acid sequence having at least 70% identity to SEQ ID NO:1.

2. The isolated nucleic acid of claim 1 encoding a polypeptide comprising the amino acid sequence of SEQ ID NO:1.

3. The isolated nucleic acid of claim 1 comprising a nucleic acid sequence having at least 70% identity to about 500 contiguous nucleotides selected from SEQ ID NO:2 or the complement thereof.

4. The isolated nucleic acid of claim 1 comprising the nucleic acid sequence of SEQ ID NO:2 or the complement thereof.

5. The isolated nucleic acid of claim 1 comprising a nucleic acid sequence having at least 70% identity to about 500 contiguous nucleotides selected from SEQ ID NO:3 or the complement thereof.

6. The isolated nucleic acid of claim 1 comprising the nucleic acid sequence of SEQ ID NO:3 or the complement thereof.

7. An isolated polypeptide comprising an amino acid sequence having at least 70% identity to SEQ ID NO:1.

8. The isolated polypeptide of claim 7, wherein the polypeptide comprises the amino acid sequence of SEQ ID NO:1.

9. An isolated polypeptide comprising an amino acid sequence having at least 70% identity to amino acids 1-81 of SEQ ID NO:1.

10. The isolated polypeptide of claim 9, wherein the polypeptide comprises amino acids 1-81 of SEQ ID NO:1.

11. An isolated oligonucleotide comprising at least 10 consecutive nucleotides of SEQ ID NO:2 or its complementary strand.

12. An isolated oligonucleotide comprising at least 10 consecutive nucleotides of SEQ ID NO:3 or its complementary strand.

13. A vector comprising the isolated nucleic acid of claim 1.

14. The vector of claim 13 comprising the nucleic acid sequence of SEQ ID NO:2.

15. The vector of claim 13 comprising the nucleic acid sequence of SEQ ID NO:3.

16. The vector of claim 13 or 14, wherein the nucleic acid is operably linked to a transcriptional regulatory sequence.

17. The vector of claim 13 or 14, wherein said vector is selected from the group comprising a plasmid, a cosmid, a virus, and a bacteriophage.

18. The vector of claim 13 or 14, wherein a polypeptide comprising SEQ ID NO:1 is expressed by a cell transformed with said vector.

19. An isolated host cell comprising the nucleic acid of claim 1.

20. An isolated host cell comprising the vector of claim 1 or 14.

21. The isolated host cell of claim 19, wherein the host cell is an adipocyte or a hepatocyte.

22. An isolated antibody that specifically binds to a polypeptide comprising an amino acid sequence of SEQ ID NO:1.

23. The antibody of claim 22, wherein the antibody is polyclonal.

24. The antibody of claim 22, wherein the antibody is monoclonal.

25. The antibody of claim 22, wherein the antibody is single chain monoclonal.

26. The antibody of claim 22, wherein the antibody is recombinant.

27. The antibody of claim 22, wherein the antibody is chimeric.

28. The antibody of claim 22, wherein the antibody is humanized.

29. The antibody of claim 22, wherein the antibody is mammalian.

30. The antibody of claim 22, wherein the antibody is human.

31. A transgenic non-human animal, which expresses a nucleic acid encoding a MO-1 polypeptide.

32. The transgenic non-human animal of claim 31, wherein the MO-1 polypeptide comprises the amino acid sequence of SEQ ID NO:1.

33. The transgenic non-human animal of claim 31, wherein the animal over- or under-expresses MO-1 polypeptide.

34. The transgenic non-human animal of claim 31, wherein the animal comprises a nucleic acid having at least 70% identity to SEQ ID NO:2 or the complement thereof.

35. The transgenic non-human animal of claim 31, wherein the animal comprises a nucleic acid having at least 70% identity to SEQ ID NO:3 or the complement thereof.

36. The transgenic non-human animal of claim 31, wherein the animal is a mammal.

37. The transgenic non-human animal of claim 31, wherein the animal is a mouse.

38. The transgenic non-human animal of claim 31, wherein the animal is a rat.

39. The transgenic non-human animal of claim 31, wherein the animal is a rabbit.

40. The transgenic non-human animal of claim 31, wherein the animal is a hamster.

41. The transgenic non-human animal of claim 31, wherein the animal is a sheep.

42. A transgenic non-human animal whose germ cells comprise a homozygous null mutation in the endogenous nucleic acid sequence encoding MO-1, wherein the mutation is created by insertion of a neomycin cassette, in reverse orientation to MO-1 transcription and wherein said mutation has been introduced into said animal by homologous recombination in an embryonic stem cell such that said animal does not express a functional MO-1 polypeptide.

43. The transgenic non-human animal of claim 42, wherein the animal is fertile and transmits said null mutation to its offspring.

44. The transgenic non-human animal of claim 42, wherein the animal is a mammal.

45. The transgenic non-human animal of claim 42, wherein the animal is a mouse.

46. The transgenic non-human animal of claim 42, wherein the animal is a rat.

47. The transgenic non-human animal of claim 42, wherein the animal is a rabbit.

48. The transgenic non-human animal of claim 42, wherein the animal is a hamster.

49. The transgenic non-human animal of claim 42, wherein the animal is a sheep.

50. A method of screening for an agent which affects MO-1 activity, comprising: a) contacting said agent to a cell that expresses a MO-1 polypeptide; and b) assessing a biological activity of the MO-1 in the cell.

51. The method of claim 50, wherein the biological activity of the MO-1 results in modulated glucose or lipid concentrations.

52. The method of claim 50, wherein the biological activity of the MO-1 results in decreased adipocyte proliferation or differentiation.

53. The method of claim 50, wherein the biological activity of the MO-1 results in altered expression of PPARgamma, aP2, SCD1, FAT/CD36, adiponectin, perilipin, GLUT4, or Leptin.

54. The method of claim 50, wherein the biological activity of the MO-1 is binding to a polypeptide that is SCP2, CYP2B6, MTO1-like or IRAP.

55. The method of claim 50, wherein the biological activity of the MO-1 results in altered gene expression.

56. A method of screening for an agent which affects MO-1 activity, comprising: a) administering said agent to the animal of claim 31; and b) assessing the animal for an alteration in metabolic function affected by said agent.

57. The method of claim 51, wherein the metabolic function is selected from the group consisting of glucose metabolism, lipid metabolism, or weight gain.

58. A method of detecting the presence of the nucleic acid of claim 1 in a sample, comprising: (a) contacting the sample with a nucleic acid that hybridizes to the nucleic acid of claim 1; and (b) determining whether the nucleic acid binds to a nucleic acid in the sample.

59. A method of detecting the presence of the nucleic acid of claim 6 in a sample, comprising: (a) contacting the sample with a nucleic acid that hybridizes to the nucleic acid of claim 6; and (b) determining whether the nucleic acid binds to a nucleic acid in the sample.

60. A composition comprising a polypeptide having an amino acid sequence that comprises SEQ ID NO:1 and a pharmaceutically acceptable carrier.

61. A composition comprising a polynucleotide encoding a polypeptide having an amino acid sequence that comprises SEQ ID NO:1 and a pharmaceutically acceptable carrier.

62. The composition of claim 61, wherein the polynucleotide comprises a nucleotide sequence of SEQ ID NO:2.

63. The composition of claim 61, wherein the polynucleotide comprises a nucleotide sequence of SEQ ID NO:3.

64. A kit comprising i) an isolated oligonucleotide comprising at least 10 consecutive nucleotides of SEQ ID NO:2 or its complementary strand and ii) a container.

65. The kit of claim 64, wherein the oligonucleotide comprises at least 15 consecutive nucleotides of SEQ ID NO:2 or its complementary strand.

66. A kit comprising i) an isolated oligonucleotide comprising at least 10 consecutive nucleotides of SEQ ID NO:3 or its complementary strand and ii) a container.

67. The kit of claim 66, wherein the oligonucleotide comprises at least 15 consecutive nucleotides of SEQ ID NO:3 or its complementary strand.

68. A method of producing a MO-1 polypeptide in a host cell comprising: i) transforming the host cell with a nucleic acid sequence encoding the MO-1 polypeptide; and ii) expressing the nucleic acid sequence so that the MO-1 polypeptide is produced by the host cell.

69. The method of claim 68, wherein the MO-1 polypeptide comprises the amino acid sequence of SEQ ID NO:1.

70. A method of treating a condition associated with MO-1, comprising administering a MO-1 polypeptide to a subject in need thereof, thereby treating the condition.

71. The method of claim 70, wherein the condition is morbid obesity or diabetes.

72. A method of increasing the weight of a subject, comprising administering a MO-1 polypeptide to a subject in need thereof, thereby increasing the weight of the subject.

73. The method of claim 72, wherein the subject is an animal.

74. The method of claim 73, wherein the animal is a chicken, turkey, cow, sheep, goat, or pig.

75. The method of claim 72, wherein the subject is a human.

76. A method of treating a condition associated with MO-1, comprising administering a nucleic acid that increases expression of a MO-1 polypeptide to a subject in need thereof, thereby treating the condition.

77. The method of claim 76, wherein the nucleic acid increases expression of MO-1 polypeptide endogenous to the subject.

78. The method of claim 76, wherein the nucleic acid encodes MO-1 polypeptide.

79. The method of claim 76, wherein the condition is morbid obesity or diabetes.
